(19) United States
(12) Patent Application Publication
(10) Pub. No.: US 2006/0002951 A1
(43) Pub. Date: Jan. 5, 2006

(54) Inhibition of the tRNALys3-primed initiation of reverse transcription in HIV-1 by APOBEC3G

(75) Inventors: Lawrence Kleiman, Montreal (CA); Shan Cen, Laval (CA); Fei Guo, Verdun (CA)

Correspondence Address: GOUDREAU GAGE DUBUC, 800 PLACE VICTORIA, SUITE 3400, MONTREAL, QUEBEC, H4Z 1E9, CA

(73) Assignee: McGill University

(21) Appl. No.: 11/129,442

(22) Filed: May 16, 2005

(30) Foreign Application Priority Data
May 14, 2004 (CA) 2,467,312

(51) Int. Cl. C12Q 1/70; A61K 39/12; C07K 14/16

(52) U.S. Cl. 424/186.1; 530/350

(57) ABSTRACT

The present invention generally relates to the field of antiviral therapy. More specifically, the present invention relates to the inhibition of the tRNA^{Lys3}-primed initiation of reverse transcription in viruses by APOBEC3G. The present invention further relates to a method of treating or preventing viral infections by inhibiting tRNA^{Lys3} annealing and/or priming on a viral genome thereby reducing viral replication. More particularly, the present invention relates to the use of APOBEC3G, fragments or derivatives thereof for treatment or prophylaxis of HIV-1 infection and related lentivirus infections.

### CROSS REFERENCE TO RELATED APPLICATIONS

This application claims priority on Canadian application no 2,467,312 filed on May 14, 2004, the content of which is herein incorporated by reference.

### FIELD OF THE INVENTION

The present invention generally relates to the field of antiviral therapy and prophylaxy. More specifically, the present invention relates to an inhibition of the tRNA^{Lys3}-primed initiation of reverse transcription in viruses by APOBEC3G. Broadly the present invention relates to means of overcoming the viral-promoting effects of Vif on viral replication.

### BACKGROUND OF THE INVENTION

Vif (virion infectivity factor) is a 190-240 amino acid protein that is encoded by all of the lentiviruses except for equine infectious anemia virus (1-12). Vif is required for HIV-1 to replicate in certain “non-permissive” cell types, such as primary T lymphocytes, macrophages and some of T-cell lines, including H9, but is not required in other “permissive” cell types, such as SupT1 and Jurkat cells (3,5,11). The ability of Vif-negative viruses to replicate in target cells is determined by the cell producing the virus (5,12). Thus, Vif-deficient viruses produced from non-permissive cells are impaired in their ability to replicate in target cells.

Non-permissive cells have been found to contain a protein called APOBEC3G (also known as CEM-15), which prevents HIV-1 replication in the absence of Vif (13). APOBEC3G belongs to an APOBEC superfamily containing at least 10 members, which share a cytidine deaminase motif (14). These include APOBEC1 and activation-induced cytidine deaminase (AID), which have been shown to deaminate C in RNA (14) and DNA (15), respectively. It is not known if APOBEC3G can edit RNA, but several reports suggest that this protein's anti-HIV-1 activity stems from its ability to form dU by deaminating dC in the first minus strand cDNA produced during HIV-1 reverse transcription (16-19). Vif-negative HIV-1 produced in non-permissive cells package APOBEC3G during assembly, while Vif-positive virions do not (13,16). cDNA synthesis is low in the target cell infected with Vif-negative viruses, and the minus strand cDNA made contains 1-2% of the cytosines mutated to uracil. This could allow for cDNA degradation by the DNA repair system. The coding strand found in double-stranded cDNA also contains an increase in G to A mutations that could also contribute to the anti-viral activity of APOBEC3G through mutant coding regions for viral proteins. Vif is able to bind to APOBEC3G (20), and can reduce both the cellular expression of APOBEC3G and its incorporation into virions (21). The reduction in cellular expression has been attributed to both inhibition of APOBEC3G translation and its degradation in the cytoplasm by Vif (22), and recent evidence suggests that Vif interacts with cytoplasmic APOBEC3G as part of a Vif-Cul5-SCF complex, resulting in the ubiquination of APOBEC3G and its degradation (23).

Enzymes similar to the human APOBEC superfamily are also encoded by the mouse and African green monkey (AGM) (20), and a mouse gene on chromosome 15 (murine CEM15) shows amino acid similarity and structural homology with human APOBEC3G (13, 24). Vif is not present in the simple retrovirus MuLV, and Vif from HIV-1 is unable to prevent encapsidation of murine APOBEC into HIV-1, whose packaging results in severe inhibition of HIV-1 replication (20). Interestingly, while murine APOBEC is incorporated into murine leukemia virus (MLV), it appears to have little effect upon this virus's replication (16, 18, 20). On the other hand, the human APOBEC3G (also termed hA3G) can inhibit the infectivity of different retroviruses including MLV, simian immunodeficiency virus (SIV), hepatitis C virus (HCV), hepatitis B virus (HBV) and equine infectious anaemia virus (EIAV) (16,18), although at lower efficiency than for lentivirus such as HIV-1.

The mechanism by which APOBEC3G is incorporated into Vif-negative HIV-1 is not clear. However, a recent paper reports that mutations in either of the two active sites of APOBEC3G inhibit deoxycytidine deaminase activity to different extents, but have the same anti-viral activity (54). This latter observation implies that deoxycytidine deaminase activity of APOBEC3G may not be the sole determinant of anti-viral activity. In any event, there remains a need to understand the mechanism by which APOBEC3G reduces viral replication and infectivity.

The use of transport polypeptides for biological targeting is well known and was adapted to many fields. The HIV Tat protein has been described to effect the delivery of molecules into the cytoplasm and nuclei of cells (International Application published on Mar. 3, 1994 as No. WO 94/04686 in the name of BIOGEN, INC.). However, the Tat transport polypeptides can not allow the delivery of molecules to HIV virions. Viral proteins such as Gag of Rous sarcoma virus and Moloney murine leukemia virus and portion of HIV-1 Gag protein have been used as carrier for incorporation of foreign antigens and enzymatic markers into retroviral particles (Wang et al., 1994, Virology, 200:524-534). However, most of the Gag protein sequences are essential for efficient viral particles assembly, thus limiting the use of such virion components as carrier.

More recently, Vpr/Vpx were used to target a molecule (e.g. protein chimeras) into HIV and related virions and shown to inhibit significantly reduce infectivity thereof (U.S. Pat. No. 5,861,161; U.S. Pat. No. 6,043,081; and U.S. Pat. No. 6,468,539B1; the contents of which are incorporated herein in their entirety). Thus, these patents provide one means to target molecules to mature HIV-1 and/or HIV-2 virions to affect their structural organization and/or functional integrity.

It would be desirable to be provided with a means to target a broader type of virions (e.g. not only HIV and related viruses). It would also be desirable to be provided with an agent which permits the targeting of chimeric molecules into not only HIV virions and related viruses but also other retroviruses, lentiviruses and non-retroviruses.

It would also be desirable to be provided with the identification of the protein interactions responsible for APOBEC3G incorporation into the mature virions such as those of HIV.

There also remains a need to provide a means to incorporate APOBEC3G into the mature HIV-1 and/or HIV-2 virions, as well as other virions by making use of the protein interactions responsible for incorporation of APOBEC3G therein, thereby affecting the functional integrity of the targeted virion.

There also remains a need to identify novel therapeutic targets that could be used to design new drugs useful in the treatment of lentivirus infection (e.g. HIV, SIV, EIAV) as well as other viruses infection such as hepatitis C virus and MLV.

The present invention seeks to meet these needs and other needs.

The present description refers to a number of documents, the content of which is herein incorporated by reference in their entirety.

### SUMMARY OF THE INVENTION

The present invention thus seeks to meet at least one of the above-mentioned needs.

Applicants demonstrate herein that the incorporation of APOBEC3G into HIV-1 requires sequences present between the two zinc coordination motifs found in this protein (amino acids 104-156; SEQ ID NO: 1) and the nucleocapsid (NC) sequence in Gag. HIV-1 Gag, alone among viral proteins, is sufficient to package APOBEC3G into Gag viral-like particles (VLPs).

Evidence is also presented that suggests that a RNA bridge between these two molecules is not involved in facilitating the Gag/APOBEC3G interaction.

Moreover, it is demonstrated that APOBEC3G prevents the proper annealing of tRNA^{Lys3} to the viral RNA genome, and also that wild-type tRNA^{Lys3} annealing and initiation of reverse transcription can be rescued with a transient exposure of the deproteinized tRNA^{Lys3}/viral RNA template to NCp7.

The present invention relates to the inhibition of retroviral replication and infectivity by APOBEC3G, fragments or derivatives thereof through the inhibition of tRNA^{Lys3} priming on viral genome. More particularly the present invention relates to the inhibition of retroviruses such as MLV, simian immunodeficiency virus (SIV), hepatitis C virus (HCV), and equine infectious anaemia virus (EIAV) (16,18) and to a non-retrovirus hepatitis B virus (HBV).

In one particular embodiment, the present invention relates to the inhibition of tRNA^{Lys3} annealing and priming on viral genome by inhibiting nucleocapsid facilitated reverse transcription. In one particular embodiment, APOBEC3G, fragments or derivatives thereof are used to treat or prevent viral infections (e.g. lentivirus, hepatitis C, MLV infections) by inhibiting replication of the virus (e.g. by inhibiting primer annealing and priming on viral genomes). In the case of HIV the priming is effected by tRNA^{Lys3}.

In a more particular embodiment, the present invention relates to APOBEC3G, fragments or derivatives thereof to target the nucleocapsid of HIV viruses to inhibit indirectly e.g. tRNA^{Lys3} annealing and priming on viral genome (or other tRNAs in the case of other viruses).

The present invention is based in part on the demonstration that APOBEC3G A3G, and notably human APOBEC3G (hA3G) (a cellular protein which can be incorporated into virions of HIV and into other virions), directly interacts with Gag, thereby providing means of targeting, incorporating, etc recombinant proteins, recombinant peptides and agents into virions. In one particular embodiment, such peptides or agents are antiviral agents. The present invention further defines the hA3G sequence responsible for its incorpororation into HIV virions (also termed the packaging domain) as amino acid region spanning amino acid residues 104-156 of hA3G (SEQ ID NO:1).

The present invention is also based on the demonstration that hA3G directly interacts with an HIV accessory protein termed Vif, which acts as a countermeasure of the virus to overcome the inhibitory activity of hA3G on viral replication (e.g. by inducing a degradation of hA3G). More particularly, the present invention is based on the demonstration that a region spanning from about amino acids 104 to about amino acid 156 of hA3G (SEQ ID NO: 21) is sufficient to enable interaction with Vif (a region also responsible for incorporation into the virion). The present invention is also based on the demonstration that the N- and C-terminal regions of hA3g can overcome in a dominant negative fashion the Vif-induced degradation of hA3G.

The present invention therefore provides the means to overcome the HIV countermeasure of Vif, by inhibiting the Vif-induced degradation of hA3G, resulting in a significant decrease in HIV replication.

Thus, peptides derived from hA3G were herein identified as novel therapeutic agents which can be used indirectly as antiviral agents (e.g. by using same as vehicles for incorporating antiviral agents into a virion, via its packaging (or incorporating) domain, or directly, by providing hA3G sequences which interact with Vif and antonize the Vif-mediated degradation of the native or recombinantly expressed hA3G.

Thus, in one aspect, the present invention relates to the inhibition of a Vif-mediated function designed to overcome the anti-viral effect of hA3G (e.g. inhibition of primer annealing and priming on the viral genome) through a degradation of hA3G or other means.

Thus, the present invention generally features novel methods of inhibiting viral replication or other metabolic cycles of virus infection.

In a further embodiment, the present invention relates to screening assays to identify compounds that modulate the interaction between hA3G and Gag (e.g. the NC portion thereof), shown herein to interact with the incorporation domain of hA3G (SEQ ID NO:1) or to identify compounds that modulate the interaction between hA3G and Vif.

In yet a further embodiment, the present invention relates to screening assays to identify compounds that inhibit the Vif-mediated degradation of hA3G.

In one particular aspect, the present invention relates to screening assays to identify compounds (e.g. peptides, pepdidomimetics, small molecules) that completely or partially inhibit the Vif-mediated degradation of hA3G, based on a use of the of hA3G-derived peptides.

In one aspect, the inhibitors of the present invention reduce or completely abolish Vif-mediated anti-hA3G biological activity. In a particular embodiment, the inhibitors of the present invention compete with natural endogenous APOBEC3G, and notably hA3G for binding to Vif. This reduces the inhibitory activity of Vif towards APOBEC3G's antiviral function and thus acts as an antiviral agent by inhibiting viral replication. For example, peptides or small molecules mimicking APOBEC3G-Vif interacting domain (e.g. SEQ ID NO:1), APOBEC3G's N-terminal or C-terminal domains (amino acids 1-156 or 157-384, respectively) can be used in accordance with the present invention. Alternatively, peptides or small molecules mimicking these domains can also be used to compete with endogenous or native APOBEC3G for the binding to Vif and/or for overcoming Vif-mediated degradation of APOBEC3G.

In one embodiment, an assay is a cell-based assay in which a cell which expresses a APOBEC3G protein or biologically active portion thereof, either natural or of recombinant origin, is contacted with a test compound and the ability of same to modulate a biological activity of APOBEC3 is determined.

In yet a further embodiment, modulators of APOBEC3G expression are identified in a method wherein a cell is contacted with a candidate compound and the expression of APOBEC3G mRNA or protein in the cell is determined. The level of expression of APOBEC3G mRNA or protein in the presence of the candidate compound is compared to the level of expression of APOBEC3G mRNA or protein in the absence of the candidate compound. The candidate compound can then be identified as a modulator of APOBEC3G expression based on this comparison. For example, when expression of APOBEC3G mRNA or protein is greater (statistically significantly greater) in the presence of the candidate compound than in its absence, the candidate compound is identified as a stimulator of APOBEC3G mRNA or protein expression. Alternatively, when expression of APOBEC3G mRNA or protein is less (statistically significantly less) in the presence of the candidate compound than in its absence, the candidate compound is identified as an inhibitor of APOBEC3G mRNA or protein expression. The level of APOBEC3G mRNA or protein expression in the cells can be determined by methods described herein or other methods known in the art for detecting APOBEC3G mRNA or protein.

In one embodiment, the screening assays of the present invention comprise 1) contacting a APOBEC3G protein, or functional variant thereof with Vif together, with a candidate compound; and 2) measuring a biological activity of APOBEC3G, or variant thereof, or measuring a biological activity of Vif in the presence of the candidate compound, wherein a compound that inhibits Vif function is selected when a APOBEC3G biological activity is significantly increased or a Vif function significiantly reduced in the presence of said candidate compound as compared to in the absence thereof.

In a related aspect, the present invention also relates to the use of any compound capable of inhibiting (antagonist, e.g. compound which reduces the phosphorylation of APOBEC3G ) or stimulating (agonist, e.g. compound which stimulates the phosphorylation of APOBEC3G ) APOBEC3G expression in a cell for the preparation of a pharmaceutical composition intended for the enhancement or stimulation of NK cells-mediated immune response including the treatment or prevention of infectious diseases and cancers.

In a further embodiment, the present invention features pharmaceutical composition comprising a compound of the present invention (e.g. peptides, peptidomemetics, small molecules, etc.) which can be chemically modified, in a pharmaceutically acceptable carrier or diluent. In another embodiment, the present invention features a method for treating or preventing a viral infections in a subject comprising administering to the subject a composition of the invention under conditions suitable for the treatment or prevention of the viral infection alone, or in conjunction with one or more therapeutic compounds.

In one embodiment, pharmaceutical compositions of the present invention comprise a specific nucleic acid sequence (e.g., encoding a mammalian APOBEC3G sequence and particularly hA3G sequence) or fragment thereof in a vector, under the control of appropriate regulatory sequences to target its expression into a specific type of cell (e.g., infected cell or cell targeted by the virus which is the subject of the antiviral treatment or prevention).

The methods of the present invention can be used for subjects with preexisting condition (e.g. already suffering from a viral infection), or subject to being exposed to or of being infected by targeting a particular virion by enabling an incorporation of an antiviral molecule inside the virion in accordance with one aspect of the invention; or by inhibiting or reducing the Vif-dependent inhibition of APOBEC3G function in accordance with another aspect of the present invention.

The compounds of the present invention include lead compounds and derivative compounds constructed so as to have the same or similar molecular structure or shape, as the lead compounds, but may differ from the lead compounds either with respect to susceptibility to hydrolysis or proteolysis (e.g. bioavailability), or with respect to their biological properties (e.g., increased affinity for Vif, or Gag, increased antagonizing effect on Vif's mediated degradation thereof).

In another embodiment, the present invention also relates to pharmaceutical compositions comprising one or more of the compounds described herein and a physiologically acceptable carrier. These pharmaceutical compositions can be in a variety of forms including oral dosage forms, topic creams, suppository, nasal spray and inhaler, as well as injectable and infusible solutions. Methods for preparing pharmaceutical composition are well known in the art as reference can be made to Remington's Pharmaceutical Sciences, Mack Publishing Company, Eaton, Pa., USA.

The compounds of the present invention can be administered to a subject to completely or partially inhibit the activity of Vif in vivo. Thus the methods of the present invention are useful in the therapeutic treatment of viral infections in which a viral protein targets APOBEC3G, in order to overcome APOBEC3G's inhibitory effect on viral replication. Of course, the compounds of the present invention may be utilized alone or in combination with any other appropriate therapies (e.g. anti-viral therapies), as determined by the practitioner.

The present invention relates to means to target molecules to mature HIV-1 and/or HIV-2 virions, as well as other virions to affect their structural organization and/or functional integrity.

The present invention also relates to an APOBEC3G protein or fragment thereof which permits the development of chimeric molecules that can be specifically targeted into mature HIV-1 and/or HIV-2 virions, as well as other virions to affect their structural organization and/or functional integrity, thereby resulting in treatment of viral infections.

In addition the present invention relates to a protein for targeting into a mature HIV-1 and/or HIV-2 virion, as well as other virions, the protein comprising a sufficient number of amino acids of APOBEC3G protein, functional derivative or fragments thereof, wherein the protein interacts with a Gag-precursor protein of the mature virion and is incorporated by the virion. More specifically, the protein interacts with the NC which is a component of the Gag-precursor protein.

More specifically, one protein of the present invention, further comprises a protein fragment covalently attached to its N or C-terminal to form a chimeric protein which is also incorporated by the mature virion. Such an attached protein fragment of the present invention consists of amino acid sequence effective in reducing HIV (or other virus) expression or replication, the amino acid sequence encoding for example an RNase activity, protease activity, creating steric hindrance during virion assembly and morphogenesis and/or affecting viral protein interactions responsible for infectivity and/or viral replication.

More specifically, the protein of the present invention, further comprises a molecule to form a protein-molecule complex which is also incorporated by the mature virion. Such a molecule is selected from the group consisting of anti-viral agents, RNases, proteases, and amino acid sequences capable of creating steric hindrance during virion assembly and morphogenesis. The molecule of the protein-molecule complex of the present invention affects the structural organization or functional integrity of the mature virion by steric hindrance or enzymatic disturbance of the virion.

The present invention further relates to a method of substantially reducing expression or replication of a virus in a patient (e.g. HIV) infected with the virus (e.g. HIV-1 and/or HIV-2), which comprises administering at least one therapeutic agent selected from the group consisting of the protein or DNA sequences encoding the protein of the present invention, to the patient in association with a pharmaceutically acceptable carrier. The administration step of the method is effected intracellularly for anti-viral treatment including gene therapy or intracellular immunization of the patient through DNA transfection or administration of the chimeric protein. The anti-viral treatment can be effected through transfection of a patient's hematopoietic cells with a DNA construct harboring a APOBEC3G chimeric protein, followed by readministration of the transfected cells, and/or through administration of a DNA construct harboring a APOBEC3G chimeric protein or directly by administration of a APOBEC3G chimeric protein, via the blood stream or otherwise.

The present invention in addition relates to a vector comprising: (a) a DNA segment encoding a protein, or peptide which enables an incorporation of a recombinant APOBEC3G construct into a virion (e.g. HIV-1 and/or HIV-2 virions), comprising a sufficient number of amino acids of an APOBEC3G protein, functional derivative or fragment thereof; and (b)a promoter upstream of the DNA segment.

In another embodiment of the present invention, there is provided a vector encoding an APOBEC3G protein, peptide or derivative which interferes with the Vif-dependent degradation of APOBEC3G, thereby protecting native APOBEC3G degradation and inhibiting viral replication (tRNA priming and annealing to the viral genome [tRNA^{Lys3}, tRNA^{Pro}, depending on the targeted virion]) ; and (b) a promoter upstream of the DNA segment.

In accordance with the present invention, two different approaches using the APOBEC3G protein and derivatives thereof are described herein for the treatment and/or prevention of viral infections.

In the first approach, APOBEC3G protein, peptide or derivative thereof is used as an inhibitor of the viral-based Vif protein (or homologs thereof), an accessory protein of HIV whose function includes a triggering of the degradation of APOBEC3G, thereby overcoming the inhibitory effect of APOBEC3G on viral replication. In accordance with this approach the supply of exogenous APOBEC3G, or derivative thereof (or increase in expression of native APOBEC3G) overcomes the inhibitory effect of Vif.

In the second approach, the incorporation domain of APOBEC3G is used to incorporate an agent into a virion.

In accordance with the second aspect of the present invention, the sequence responsible for virion targeting, incorporation and the like is termed herein the APOBEC3G incorporation domain.

The expression “functional fragments or derivatives of the incorporation domain” when used herein is intended to mean any substitutions, deletions and/or additions of amino acids that do not negatively affect the virion incorporation function of the APOBEC3G incorporation domain.

In accordance with the second approach of the present invention, an APOBEC3G chimeric protein comprises an amino acid sequence of a APOBEC3G protein or a functional derivative thereof and a molecule attached to the amino acid sequence. The molecule may be covalently attached at the N- or C-terminal of the amino acid sequence or it may be attached to the amino acid sequence at any amino acid position by chemical cross-linking or by genetic fusion.

A preferred molecule used in accordance with the present invention may be selected from the group consisting of an anti-viral agent and/or a second amino acid sequence which contains a sufficient number of amino acids corresponding to RNases, proteases, or any protein capable of creating steric hindrance during virion morphogenesis and/or affecting viral protein interactions responsible for infectivity and/or viral replication.

The APOBEC3G protein in accordance with the second approach of the present invention may be used for the targeting of molecules into the mature virions of HIV-1 and/or HIV-2, for example, such as polypeptides, proteins and anti-viral agents, among others.

The treatment in accordance with the present invention may consist in achieving the production of viral particles having substantially reduced replication capacity.

In order to provide a clear and consistent understanding of terms used in the specification and claims, including the scope to be given such terms, a number of definitions are provided herein below.

### DEFINITIONS

Unless defined otherwise, the scientific and technological terms and nomenclature used herein have the same meaning as commonly understood by a person of ordinary skill to which this invention pertains. Commonly understood definitions of molecular biology terms can be found for example in Dictionary of Microbiology and Molecular Biology, 2nd ed. (Singleton et al., 1994, John Wiley & Sons, New York, N.Y.), The Harper Collins Dictionary of Biology (Hale & Marham, 1991, Harper Perennial, New York, N.Y.), Rieger et al., Glossary of genetics: Classical and molecular, 5^{th} edition, Springer-Verlag, New-York, 1991; Alberts et al., Molecular Biology of the Cell, 4^{th} edition, Garland science, New-York, 2002; and, Lewin, Genes VII, Oxford University Press, New-York, 2000. Generally, the methods traditionally used in molecular biology, such as preparative extractions of plasmid DNA, centrifugation of plasmid DNA in cesium chloride gradient, agarose or acrylamide gel electrophoresis, purification of DNA fragments by electroelution, phenol or pheol-chloroform extraction of proteins, ethanol or isopropanol precipitation of DNA in saline medium, transformation into bacteria or transfection into cells, procedure for cell culture, infection, methods and the like are common methods used in the art. Such standard techniques can be found in reference manuals such as for example Sambrook et al. (2000, Molecular Cloning—A Laboratory Manual, Third Edition, Cold Spring Harbor Laboratories); and Ausubel et al. (1994, Current Protocols in Molecular Biology, John Wiley & Sons, New-York). In addition, methods and procedures to produce transgenic animals are well-known in the art and described in details for example in: Hogan et al., 1994, Manipulating the Mouse Embryo, Cold Spring Harbor Laboratory Press; Nagy et al., 2002, Manipulating the Mouse Embryo, 3rd edition, Cold Spring Harbor Laboratory Press.

The use of the word “a” or “an” when used in conjunction with the term “comprising” in the claims and/or the specification may mean “one” but it is also consistent with the meaning of “one or more”, “at least one”, and “one or more than one”.

Throughout this application, the term “about” is used to indicate that a value includes the standard deviation of error for the device or method being employed to determine the value. In general, the terminology “about” is meant to designate a possible variation of up to 10%. Therefore, a variation of 1, 2, 3, 4, 5, 6, 7, 8, 9 and 10% of a value is included in the term about.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, un-recited elements or method steps.

The abbreviations used include: HIV-1, human immunodeficiency virus type 1; BH10P-, HIV-1 containing an inactive viral protease; PAGE, polyacrylamide gel electrophoresis; RT, reverse transcriptase; Gag, HIV-1 precursor protein containing sequences coding for HIV-1 structural proteins: MA, matrix; CA, capsid; NC, nucleocapsid; p6, p6 protein; VLP, viral-like-particle; Vif, viral infectivity factor; HA, hemagglutinin epitope.

Nucleotide sequences are presented herein by single strand, in the 5′ to 3′ direction, from left to right, using the one-letter nucleotide symbols as commonly used in the art and in accordance with the recommendations of the IUPAC IUB Biochemical Nomenclature Commission.

As used herein, “nucleic acid molecule” or “polynucleotides”, refers to a polymer of nucleotides. Non-limiting examples thereof include DNA (e.g. genomic DNA, cDNA), RNA molecules (e.g. mRNA) and chimeras thereof. The nucleic acid molecule can be obtained by cloning techniques or synthesized. DNA can be double-stranded or single-stranded (coding strand or non-coding strand [antisense]). Conventional ribonucleic acid (RNA) and deoxyribonucleic acid (DNA) are included in the terms “nucleic acid” and “polynucleotides” as are analogs thereof. A nucleic acid backbone may comprise a variety of linkages known in the art, including one or more of sugar-phosphodiester linkages, peptide-nucleic acid bonds (referred to as “peptide nucleic acids” (PNA); Hydig-Hielsen et al., PCT Int'l Pub. No. WO 95/32305), phosphorothioate linkages, methylphosphonate linkages or combinations thereof. Sugar moieties of the nucleic acid may be ribose or deoxyribose, or similar compounds having known substitutions, e.g., 2′ methoxy substitutions (containing a 2′-O-methylribofuranosyl moiety; see PCT No. WO 98/02582) and/or 2′ halide substitutions. Nitrogenous bases may be conventional bases (A, G, C, T, U), known analogs thereof (e.g., inosine or others; see The Biochemistry of the Nucleic Acids 5-36, Adams et al., ed., 11th ed., 1992), or known derivatives of purine or pyrimidine bases (see, Cook, PCT Int'l Pub. No. WO 93/13121) or “abasic” residues in which the backbone includes no nitrogenous base for one or more residues (Arnold et al., U.S. Pat. No. 5,585,481). A nucleic acid may comprise only conventional sugars, bases and linkages, as found in RNA and DNA, or may include both conventional components and substitutions (e.g., conventional bases linked via a methoxy backbone, or a nucleic acid including conventional bases and one or more base analogs).

The terminology “APOBEC3G nucleic acid” or “APOBEC3G polynucleotide” refers to a native APOBEC3G nucleic acid sequence. In one embodiment, the human APOBEC3G sequence has the sequences set forth in SEQ ID NOs: 20 and 21 and schematized in FIGS. 4, 11 and 12 as well as in FIG. 17. In view of the conservation of the sequences as shown in FIG. 17, but also of some of the differences it is clear that some modifications to the sequences can be effected without compromising the functional activity of APOBEC3G. Such modifications are also within the scope of the present invention.

An “isolated nucleic acid molecule”, as is generally understood and used herein, refers to a polymer of nucleotides, and includes but should not be limited to DNA and RNA. The “isolated” nucleic acid molecule is purified from its natural in vivo state.

By “RNA” or “mRNA” is meant a molecule comprising at least one ribonucleotide residue. By ribonucleotide is meant a nucleotide with a hydroxyl group at the 2′ position of a β-D-ribo-furanose moiety. The term include double stranded RNA, single stranded RNA, isolated RNA such as partially purified RNA, essentially purified RNA, synthetic RNA, recombinantly produced RNA, as well as altered RNA that differs from naturally occurring RNA by the addition, deletion, substitution and/or alteration of one or more nucleotide. Such alterations can include addition of non-nucleotide material, such as to the end(s) of a siRNA or internally, for example at one or more nucleotides of the RNA molecule. Nucleotides in the RNA molecules of the instant invention can also comprise non-standard nucleotides or chemically synthesized nucleotides or deoxynucleotides. These altered RNAs can be referred to as analogs or analogs of naturally occurring RNA.

Complementary DNA (cDNA). Recombinant nucleic acid molecules synthesized by reverse transcription of messenger RNA (“mRNA”).

Expression. By the term “expression” is meant the process by which a gene or otherwise nucleic acid sequence produces a polypeptide. It involves transcription of the gene into mRNA, and the translation of such mRNA into polypeptide(s).

The term “vector” is commonly known in the art and defines a plasmid DNA, phage DNA, viral DNA and the like, which can serve as a DNA vehicle into which nucleic acid of the present invention can be cloned. Numerous types of vectors exist and are well known in the art. One specific type of vector is called a targeting vector which may be used for homologous recombination with an endogenous target gene in a cell. Homologous recombination occurs between two sequences (i.e. the targeting vector and endogenous gene sequences) that are partially or fully complementary. Homologous recombination may be used to alter a gene sequence in a cell (e.g. embryonic stem cells, (ES cells)) in order to completely shut down protein expression or to introduce point mutations, substitutions or deletions in the target gene sequence. Such method is used for example to generate transgenic animals and is well known in the art.

Expression Vector. A vector or vehicle similar to a cloning vector but which is capable of expressing a gene which has been cloned into it, after transformation into a host. The cloned gene (or nucleic acid sequence) is usually placed under the control of (i.e., operably linked to) certain control sequences such as promoter sequences which may be cell or tissue specific (e.g. innate immune cells).

Expression control sequences will vary depending on whether the vector is designed to express the operably linked gene (or nucleic acid sequence) in a prokaryotic and/or eukaryotic host and can additionally contain transcriptional elements such as enhancer elements, termination sequences, tissue-specificity elements, and/or translational initiation and termination sites. Vectors which can be used both in prokaryotic and eukaryotic cells are often called shuttle vectors. In particular embodiment, the control sequences may allow general expression (i.e. expression in a large number of cell types) or tissue specific or cell specific expression of a particular nucleic acid sequence ( e.g. in innate immune cells).

A DNA construct can be a vector comprising a promoter that is operably linked to an oligonucleotide sequence of the present invention, which is in turn, operably linked to a heterologous gene, such as the gene for the luciferase reporter molecule. “Promoter” refers to a DNA regulatory region capable of binding directly or indirectly to RNA polymerase in a cell and initiating transcription of a downstream (3′ direction) coding sequence. For purposes of the present invention, the promoter is bound at its 3′ terminus by the transcription initiation site and extends upstream (5′ direction) to include the minimum number of bases or elements necessary to initiate transcription at levels detectable above background. Within the promoter will be found a transcription initiation site (conveniently defined by mapping with S1 nuclease), as well as protein binding domains (consensus sequences) responsible for the binding of RNA polymerase. Eukaryotic promoters will often, but not always, contain “TATA” boxes and “CCAT” boxes. Prokaryotic promoters contain Shine Dalgarno sequences in addition to the −10 and −35 consensus sequences.

As used herein, the term “gene therapy” relates to the introduction and expression in an animal (preferably a human) of an exogenous sequence (e.g., a APOBEC3G gene or cDNA sequence or part thereof or derivative thereof), to supplement a native APOBEC3G sequence, inhibit a target gene (i.e., Vif), to enable target cells to produce a protein (e.g., an APOBEC3G protein, part thereof or derivative chimeric protein to target a specific virion) having a prophylactic or therapeutic effect toward viral diseases.

Nucleic acid sequences may be detected by using hybridization with a complementary sequence (e.g., oligonucleotide probes—see U.S. Pat. No. 5,503,980 (Cantor); U.S. Pat. No. 5,202,231 (Drmanac et al.); U.S. Pat. No. 5,149,625 (Church et al.); U.S. Pat. No. 5,112,736 (Caldwell et al.); U.S. Pat. No. 5,068,176 (Vijg et al.); and U.S. Pat. No. 5,002,867 (Macevicz)). Hybridization detection methods may use an array of probes (e.g., on a DNA chip) to provide sequence information about the target nucleic acid which selectively hybridizes to an exactly complementary probe sequence in a set of four related probe sequences that differ by one nucleotide (see U.S. Pat. Nos. 5,837,832 and 5,861,242 (Chee et al.). In addition, any other well known hybridization technique (Northern blot, dot blot, Southern blot) may be used in accordance with the present invention.

Nucleic Acid Hybridization. Nucleic acid hybridization depends on the principle that two single-stranded nucleic acid molecules that have complementary base sequences will reform the thermodynamically favored double-stranded structure if they are mixed under the proper conditions. The double-stranded structure will be formed between two complementary single-stranded nucleic acids even if one is immobilized on a nitrocellulose filter. In the Southern or Northern hybridization procedures, the latter situation occurs. The DNA/RNA of the individual to be tested may be digested with a restriction endonuclease if applicable, prior to its fractionation by agarose gel electrophoresis, conversion to the single-stranded form, and transfer to nitrocellulose paper, making it available for reannealing to the hybridization probe. Non-limiting examples of hybridization conditions can be found in Ausubel, F. M. et al., Current protocols in Molecular Biology, John Wiley & Sons, Inc., New York, N.Y. (1994). For purposes of illustration, an example of moderately stringent conditions for testing the hybridization of a polynucleotide of the present invention with other polynucleotides, include prewashing, in a solution of 5×SSC, 0.5% SDS, 1 mM EDTA (pH 8.0); hybridizing at 50° C.-60° C., 5×SSC and 100 μg/ml denatured salmon sperm DNA overnight (12-16 hours); followed by washing twice at 60° C. for 15 minutes with each of 2×SSC, 0.5×SSC and 0.2×SSC containing 0.1% SDS. For example for highly stringent hybridization conditions, the hybridization temperature is changed to 62, 63, 64, 65, 66, 67 or 68° C. One skilled in the art will understand that the stringency of hybridization can be readily manipulated, such as by altering the salt and SDS concentration of the hybridizing and washing solutions and/or temperature at which the hybridization is performed. The temperature and salt concentration selected is determined based on the melting temperature (Tm) of the DNA hybrid. Other protocols or commercially available hybridization kits using different annealing and washing solutions can also be used as well known in the art. The use of formamide in different mixtures to lower the melting temperature may also be used and is well known in the art.

A “probe” is meant to include a nucleic acid oligomer that hybridizes specifically to a target sequence in a nucleic acid or its complement, under conditions that promote hybridization, thereby allowing detection of the target sequence or its amplified nucleic acid. Detection may either be direct (i.e, resulting from a probe hybridizing directly to the target or amplified sequence) or indirect (i.e., resulting from a probe hybridizing to an intermediate molecular structure that links the probe to the target or amplified sequence). A probe's “target” generally refers to a sequence within an amplified nucleic acid sequence (i.e., a subset of the amplified sequence) that hybridizes specifically to at least a portion of the probe sequence by standard hydrogen bonding or “base pairing.”

By “sufficiently complementary” is meant a contiguous nucleic acid base sequence that is capable of hybridizing to another sequence by hydrogen bonding between a series of complementary bases. Complementary base sequences may be complementary at each position in sequence by using standard base pairing (e.g., G:C, A:T or A:U pairing) non standard base pairing (e.g., I:C) or may contain one or more residues (including a basic residues) that are not complementary by using standard base pairing, but which allow the entire sequence to specifically hybridize with another base sequence in appropriate hybridization conditions. Contiguous bases of an oligomer are preferably at least about 80% (81, 82, 83, 84, 85, 86, 87, 88, 89, 90, 91, 92, 93, 94, 95, 96, 97, 98, 99, 100%), more preferably at least about 90% complementary to the sequence to which the oligomer specifically hybridizes. Determination of binding free energies for nucleic acid molecules is well known in the art (e.g., see Turner et al., 1987, J. Am. Chem. Soc. 190:3783-3785; Frier et al., 1986 Proc. Nat. Acad. Sci. USA, 83: 9373-9377).

“Perfectly complementary” means that all the contiguous residues of a nucleic acid molecule will hydrogen bond with the same number of contiguous residues in a second nucleic acid sequence. Appropriate hybridization conditions are well known to those skilled in the art, can be predicted readily based on sequence composition and conditions, or can be determined empirically by using routine testing (see Sambrook et al., (cf. Molecular Cloning: A Laboratory Manual, Third Edition, edited by Cold Spring Harbor Laboratory, 2000) at §§ 1.90-1.91, 7.37-7.57, 9.47-9.51 and 11.47-11.57, particularly at §§ 9.50-9.51, 11.12-11.13, 11.45-11.47 and 11.55-11.57). Sequences that are “sufficiently complementary” allow stable hybridization of a probe sequence to a target sequence, even if the two sequences are not completely identical.

A detection step may use any of a variety of known methods to detect the presence of nucleic acid by hybridization to a probe oligonucleotide. One specific example of a detection step uses a homogeneous detection method such as described in detail previously in Arnold et al. Clinical Chemistry 35:1588-1594 (1989), and U.S. Pat. No. 5,658,737 (Nelson et al.), and U.S. Pat. Nos. 5,118,801 and 5,312,728 (Lizardi et al.).

The types of detection methods in which probes can be used include Southern blots (DNA detection), dot or slot blots (DNA, RNA), and Northern blots (RNA detection). Labeled proteins could also be used to detect a particular nucleic acid sequence to which it binds (e.g protein detection by far western technology: Guichet et al., 1997, Nature 385(6616): 548-552; and Schwartz et al., 2001, EMBO 20(3): 510-519). Other detection methods include kits containing reagents of the present invention on a dipstick setup and the like. Of course, it might be preferable to use a detection method which is amenable to automation. A non-limiting example thereof includes a chip or other support comprising one or more (e.g. an array) different probes.

A “label” refers to a molecular moiety or compound that can be detected or can lead to a detectable signal. A label is joined, directly or indirectly, to a nucleic acid probe or the nucleic acid to be detected (e.g., an amplified sequence). Direct labeling can occur through bonds or interactions that link the label to the nucleic acid (e.g., covalent bonds or non-covalent interactions), whereas indirect labeling can occur through the use of a “linker” or bridging moiety, such as additional oligonucleotide(s), which is either directly or indirectly labeled. Bridging moieties may amplify a detectable signal. Labels can include any detectable moiety (e.g., a radionuclide, ligand such as biotin or avidin, enzyme or enzyme substrate, reactive group, chromophore such as a dye or colored particle, luminescent compound including a bioluminescent, phosphorescent or chemiluminescent compound, and fluorescent compound). In one particular embodiment, the label on a labeled probe is detectable in a homogeneous assay system, i.e., in a mixture, the bound label exhibits a detectable change compared to an unbound label.

Other methods of labeling nucleic acids are known whereby a label is attached to a nucleic acid strand as it is fragmented, which is useful for labeling nucleic acids to be detected by hybridization to an array of immobilized DNA probes (e.g., see PCT No. PCT/IB99/02073).

As used herein, “oligonucleotides” or “oligos” define a molecule having two or more nucleotides (ribo or deoxyribonucleotides). The size of the oligo will be dictated by the particular situation and ultimately on the particular use thereof and adapted accordingly by the person of ordinary skill. An oligonucleotide can be synthesized chemically or derived by cloning according to well-known methods. While they are usually in a single-stranded form, they can be in a double-stranded form and even contain a “regulatory region”. They can contain natural, rare or synthetic nucleotides. They can be designed to enhance a chosen criterion like stability, for example. Chimeras of deoxyribonucleotides and ribonucleotides may also be within the scope of the present invention.

“Amplification” refers to any known in vitro procedure for obtaining multiple copies (“amplicons”) of a target nucleic acid sequence or its complement or fragments thereof. In vitro amplification refers to the production of an amplified nucleic acid that may contain less than the complete target region sequence or its complement. Known in vitro amplification methods include, e.g., transcription mediated amplification, replicase-mediated amplification, polymerase chain reaction (PCR) amplification, ligase chain reaction (LCR) amplification, nucleic acid sequence-based amplification (NASBA), and strand-displacement amplification (SDA). Replicase-mediated amplification uses self-replicating RNA molecules, and a replicase such as Qβg-replicase (e.g., Kramer et al., U.S. Pat. No. 4,786,600). PCR amplification is well known and uses DNA polymerase, primers and thermal cycling to synthesize multiple copies of the two complementary strands of DNA or cDNA (e.g., Mullis et al., U.S. Pat. Nos. 4,683,195, 4,683,202, and 4,800,159). LCR amplification uses at least four separate oligonucleotides to amplify a target and its complementary strand by using multiple cycles of hybridization, ligation, and denaturation (e.g., EP Pat. App. Pub. No. 0 320 308). SDA is a method in which a primer contains a recognition site for a restriction endonuclease that permits the endonuclease to nick one strand of a hemimodified DNA duplex that includes the target sequence, followed by amplification in a series of primer extension and strand displacement steps (e.g., Walker et al., U.S. Pat. No. 5,422,252). Another known strand-displacement amplification method does not require endonuclease nicking (Dattagupta et al., U.S. Pat. No. 6,087,133). Transcription-mediated amplification (TMA) can also be used in the present invention. In one embodiment, TMA and NASBA isothermic methods of nucleic acid amplification are used. Those skilled in the art will understand that the oligonucleotide primer sequences of the present invention may be readily used in any in vitro amplification method based on primer extension by a polymerase (see generally Kwoh et al., 1990, Am. Biotechnol. Lab. 8:14 25 and (Kwoh et al., 1989, Proc. Natl. Acad. Sci. USA 86, 1173 1177; Lizardi et al., 1988, BioTechnology 6:1197 1202; Malek et al., 1994, Methods Mol. Biol., 28:253 260; and Sambrook et al., (cf. Molecular Cloning: A Laboratory Manual, Third Edition, edited by Cold Spring Harbor Laboratory, 2000). As commonly known in the art, the oligos are designed to bind to a complementary sequence under selected conditions.

As used herein, a “primer” defines an oligonucleotide which is capable of annealing to a target sequence, thereby creating a double stranded region which can serve as an initiation point for nucleic acid synthesis under suitable conditions. Primers can be, for example, designed to be specific for certain alleles so as to be used in an allele-specific amplification system. The primer's 5′ region may be non-complementary to the target nucleic acid sequence and include additional bases, such as a promoter sequence (which is referred to as a “promoter primer”). Those skilled in the art will appreciate that any oligomer that can function as a primer can be modified to include a 5′ promoter sequence, and thus function as a promoter primer. Similarly, any promoter primer can serve as a primer, independent of its functional promoter sequence. Of course the design of a primer from a known nucleic acid sequence is well known in the art. As for the oligos, it can comprise a number of types of different nucleotides.

As used herein, the twenty natural amino acids and their abbreviations follow conventional usage. Stereoisomers (e.g., D-amino acids) such as a,a-disubstituted amino acids, N-alkyl amino acids, lactic acid and other unconventional amino acids may also be suitable components for the polypeptides of the present invention. Examples of unconventional amino acids include but are not limited to selenocysteine, citrulline, ornithine, norvaline, 4-(E)-butenyl-4(R)-methyl-N-methylthreonine (MeBmt), N-methyl-leucine (MeLeu), aminoisobutyric acid, statine, N-methyl-alanine (MeAla).

As used herein, “protein” or “polypeptide” means any peptide-linked chain of amino acids, regardless of post-translational modifications (e.g. acetylation, phosphorylation, glycosylation, sulfatation, sumoylation, prenylation, ubiquitination, etc). An “APOBEC3G protein” or a “APOBEC3G polypeptide” is an expression product of APOBEC3G nucleic acid (e.g. APOBEC3G gene) such as native human APOBEC3G protein (FIG. 17), or a APOBEC3G protein homolog (e.g. mouse or primate APOBEC3G APOBEC3G, FIG. 17) that shares at least 60% (but preferably, at least 65, 70, 75, 80, 85, 86, 87, 88, 89, 90, 91, 92, 93, 94, 95, 96, 97, 98, 99, 100%) amino acid sequence identity with APOBEC3G and displays functional activity of native APOBEC3G protein. For the sake of brevity, the units (e.g. 66, 67 . . . 81, 82% . . . ) have not been specifically recited but are nevertheless considered within the scope of the present invention.

An “APOBEC3G interacting protein” refers to a protein which binds directly or indirectly (e.g. via RNA) to APOBEC3G (e.g. Vif, Gag etc.) in order to modulate or participate in a functional activity of APOBEC3G and/or to modulate an activity of Vif and/or Gag.

The terms “biological activity” or “functional activity” or “function” are used interchangeably and refer to any detectable biological activity associated with a structural, biochemical or physiological activity of a cell or protein (i.e. APOBEC3G). For instance, one non-limiting example of a functional activity of APOBEC3G protein includes interacting with Vif. Another is interacting with Gag (e.g. NC). Yet another is being incorporated in a mature virion (e.g. HIV-1). Other domains (e.g. other than the sequences that interact with Vif and/or Gag) of APOBEC3G are described and shown in the Figures. In any event, interaction of APOBEC3G with any of the APOBEC3G interacting proteins is considered a functional activity of an APOBEC3G protein. Such interaction may be stable or transient. Another example of an APOBEC3G functional activity is its function on annealing or priming by a particular tRNA. Thus, in accordance with the present invention, measuring the effect of a test compound on its ability to inhibit or increase (e.g., modulate) APOBEC3G binding or interaction, level of expression as well as replication inhibition, incorporation into virions, etc. is considered herein as measuring a biological activity of APOBEC3G.

As noted above, APOBEC3G biological activity also includes any biochemical measurement of the protein, conformational changes, phosphorylation status (or any other posttranslational modification e.g. ubiquitination, etc), or any other feature of the protein that can be measured with techniques known in the art.

As used herein, the designation “functional derivative” denotes, in the context of a functional derivative of an amino acid sequence, a molecule that retains a biological activity (either function or structural) that is substantially similar to that of the original sequence. This functional derivative or equivalent may be a natural derivative or may be prepared synthetically. Such derivatives include amino acid sequences having substitutions, deletions, or additions of one or more amino acids, provided that the biological activity of the protein is conserved. The substituting amino acid generally has chemico-physical properties, which are similar to that of the substituted amino acid. The similar chemico-physical properties include, similarities in charge, bulkiness, hydrophobicity, hydrophylicity and the like. The term “functional derivatives” is intended to include “segments”, “variants”, “analogs” or “chemical derivatives” of the subject matter of the present invention.

As used herein, “chemical derivatives” is meant to cover additional chemical moieties not normally part of the subject matter of the invention. Such moieties could affect the physico chemical characteristic of the derivative (i.e. solubility, absorption, half life and the like, decrease of toxicity). Such moieties are exemplified in Remington: The Science and Practice of Pharmacy by Alfonso R. Gennaro, 2003, 21th edition, Mack Publishing Company. Methods of coupling these chemical physical moieties to a polypeptide are well known in the art.

As used herein, the term “pharmaceutically acceptable” refers to molecular entities and compositions that are physiologically tolerable and do not typically produce an allergic or similar untoward reaction, such as gastric upset, dizziness and the like, when administered to human. Preferably, as used herein, the term “pharmaceutically acceptable” means approved by regulatory agency of the federal or state government or listed in the U.S. Pharmacopeia or other generally recognized pharmacopeia for use in animals, and more particularly in humans. The term “carrier” refers to a diluent, adjuvant, excipient, or vehicle with which the compounds of the present invention may be administered. Sterile water or aqueous saline solutions and aqueous dextrose and glycerol solutions may be employed as carrier, particularly for injectable solutions. Suitable pharmaceutical carriers are described in “Remington's Pharmaceutical Sciences” by E. W. Martin.

As commonly known, a “mutation” is a detectable change in the genetic material which can be transmitted to a daughter cell. As well known, a mutation can be, for example, a detectable change in one or more deoxyribonucleotide. For example, nucleotides can be added, deleted, substituted for, inverted, or transposed to a new position. Spontaneous mutations and experimentally induced mutations exist. The result of a mutation of nucleic acid molecule is a mutant nucleic acid molecule. A mutant polypeptide can be encoded from this mutant nucleic acid molecule.

The term “variant” refers herein to a protein, which is substantially similar in structure and biological activity to the protein, or nucleic acid of the present invention to maintain at least one of its biological activities. Thus, provided that two molecules possess a common activity and can substitute for each other, they are considered variants as that term is used herein, even if the composition, or secondary, tertiary or quaternary structure of one molecule is not identical to that found in the other, or if the amino acid sequence or nucleotide sequence is not identical. A homolog is a gene sequence encoding a polypeptide isolated from an organism other than a human being. Similarly, a homolog of a native polypeptide is an expression product of a gene homolog. Expression vectors, regulatory sequences (e.g. promoters), leader sequences and method to generate same and introduce them in cells are well known in the art.

Binding agent. A binding agent is a molecule or compound that specifically binds to or interacts with a APOBEC3G or polypeptide. Non-limiting examples of binding agents include antibodies, interacting partners, ligands, and the like. It will be understood that such binding agents can be natural, recombinant or synthetic.

In accordance with the present invention, it shall be understood that the “in vivo” experimental model can also be used to carry out an “in vitro” assay. For example, cellular extracts from the indicator cells can be prepared and used in one of the aforementioned “in vitro” tests (such as in binding assays or in vitro translation assays).

The term “subject” or “patient” as used herein refers to an animal, preferably a mammal, most preferably a human who is the object of treatment, observation or experiment.

As used herein, the term “purified” refers to a molecule (e.g. APOBEC3G polypeptides, etc) having been separated from a component of the composition in which it was originally present. Thus, for example, a “purified APOBEC3G polypeptide or polynucleotide” has been purified to a level not found in nature. A “substantially pure” molecule is a molecule that is lacking in most other components (e.g., 30, 40, 50, 60, 70, 75, 80, 85, 90, 95, 96, 97, 98, 99, 100% free of contaminants). By opposition, the term “crude” means molecules that have not been separated from the components of the original composition in which it was present. Therefore, the terms “separating” or “purifying” refers to methods by which one or more components of the biological sample are removed from one or more other components of the sample. Sample components include nucleic acids in a generally aqueous solution that may include other components, such as proteins, carbohydrates, or lipids. A separating or purifying step preferably removes at least about 70% (e.g., 70, 75, 80, 85, 90, 95, 96, 97, 98, 99, 100%), more preferably at least about 90% (e.g., 90, 91, 92, 93, 94, 95, 96, 97, 98, 99, 100%) and, even more preferably, at least about 95% (e.g., 95, 96, 97, 98, 99, 100%) of the other components present in the sample from the desired component. For the sake of brevity, the units (e.g. 66, 67 . . . 81, 82, . . . 91, 92% . . . ) have not systematically been recited but are considered, nevertheless, within the scope of the present invention.

The terms “inhibiting,” “reducing” or any variation of these terms, when used in the claims and/or the specification includes any measurable decrease or complete inhibition of at least one biological activity of APOBEC3G to achieve a desired result. For example, a compound is said to be inhibiting Vif-mediated APOBEC3G activity when a decrease in viral replication, viral production, etc. is measured following a treatment with the compounds of the present invention as compared to in the absence thereof. Another non-limiting example includes a reduction in the priming or annealing of tRNA^{Lys3} on viral genome (e.g. HIV RNA).

As used herein, the terms “molecule”, “compound”, “agent” or “ligand” are used interchangeably and broadly to refer to natural, synthetic or semi-synthetic molecules or compounds. The term “compound” therefore denotes for example chemicals, macromolecules, cell or tissue extracts (from plants or animals) and the like. Non-limiting examples of compounds include peptides, antibodies, carbohydrates, nucleic acid molecules and pharmaceutical agents. The compound can be selected and screened by a variety of means including random screening, rational selection and by rational design using for example a peptide sequence of APOBEC3G in accordance with the present invention a protein or ligand modeling methods such as computer modeling.

The terms “rationally selected” or “rationally designed” are meant to define compounds which have been chosen based on the configuration of interacting domains of the present invention. As will be understood by the person of ordinary skill, macromolecules having non-naturally occurring modifications are also within the scope of the term “molecule”. For example, the modulating compounds of the present invention are modified to enhance their stability and their bioavailability. The compounds or molecules identified in accordance with the teachings of the present invention have a therapeutic value in diseases or conditions in which the physiology or homeostasis of the cell and/or tissue is compromised by a viral infection.

As used herein “antagonists”, “Vif antagonists” or “Vif inhibitors” refer to any molecule or compound capable of inhibiting (completely or partially) a biological activity of Vif.

When referring to nucleic acid molecules, proteins or polypeptides, the term native refers to a naturally occurring nucleic acid or polypeptide. A homolog is a gene sequence encoding a polypeptide isolated from an organism other than a human being. Similarly, a homolog of a native polypeptide is an expression product of a gene homolog. Of course, the non-coding portion of a gene can also find a homolog portion in another organism.

Gene Therapy Methods

In accordance with the gene therapy methods aspect of the present invention an exogenous sequence (e.g., a APOBEC3G gene or cDNA sequence), is introduced and expressed in an animal (preferably a human) to supplement, replace or provide APOBEC3G, a portion or derivative thereof to inhibit Vif function or to target virions to produce a protein (e.g., a APOBEC3G chimeric protein to target a specific molecule to the virions) having a prophylactic or therapeutic effect toward viral diseases.

Non virus-based and virus-based vectors (e.g., adenovirus- and lentivirus-based vectors) for insertion of exogenous nucleic acid sequences into eukaryotic cells are well known in the art and may be used in accordance with the present invention. Virus-based vectors (and their different variations) for use in gene therapy are well known in the art. In virus-based vectors, parts of a viral gene are replaced by the desired exogenous sequence so that a viral vector is produced. Viral vectors are no longer able to replicate due to DNA manipulations.

In one specific embodiment, lentivirus derived vectors are used to target a APOBEC3G sequence (nucleic acid encoding a partial or complete APOBEC3G protein, chimera thereof, etc.) into specific target cells. These vectors have the advantage of infecting quiescent cells (for example see U.S. Pat. No. 6,656,706; Amado et al., 1999, Science 285: 674-676).

One way of performing gene therapy is to extract cells from a patient, infect the extracted cells with a viral vector and reintroduce the cells back into the patient. A selectable marker may or may not be included to provide a means for enriching for infected or transduced cells. Alternatively, vectors for gene therapy that are specially formulated to reach and enter target cells may be directly administered to a patient (e.g., intravenously, orally etc.).

The exogenous sequences (e.g. an APOBEC3G sequence, or APOBEC3G targeting vector) may be delivered into target cells according to well known methods. Apart from infection with virus-based vectors, examples of methods to deliver nucleic acid into cells include DEAE dextran lipid formulations, liposome-mediated transfection, CaCl₂-mediated transfection, electroporation or using a gene gun. Synthetic cationic amphiphilic substances, such as dioleoyloxypropylmethylammonium bromide (DOTMA) in a mixture with dioleoylphosphatidylethanolamine (DOPE), or lipopolyamine (Behr, Bioconjugate Chem., 1994 5:382), have gained considerable importance in charged gene transfer. Due to an excess of cationic charge, the substance mixture complexes with negatively charged genes and binds to the anionic cell surface. Other methods include linking the exogenous oligonucleotide sequence (e.g., APOBEC3G sequence encoding a APOBEC3G protein, APOBEC3G targeting vector, etc) to peptides or antibodies that especially binds to receptors or antigens at the surface of a target cell. A method using non-viral carriers that are cationized to enable them to complex with the negatively charged DNA has been described U.S. Pat. No. 6,358,524. Moreover, the method also includes the use of a ligand that can specifically bind to the desired target cell in order to enter it.

Assays to Identify Modulators of APOBEC3G and Vif and/or Gag Interaction

In order to identify modulators of APOBEC3G Vif and/or Gag interaction several screening assays aiming at stimulating a functional activity of APOBEC3G in cells can be designed in accordance with the present invention.

One possible way is by screening libraries of candidate compounds for stimulators of APOBEC3G-Gag and APOBEC3G-Vif interactions. Other possibilities include screening for compounds that inhibit the APOBEC3G-dependent inhibition of Vif-dependent degradation of APOBEC3G. Inhibitors of other APOBEC3G functional activities may also be identified in accordance with the present invention, as long as such functional activities are related to APOBEC3G functions in viral replication or the viral life cycle. Screening assays and compounds which directly or indirectly modulate (i.e. decrease or increase) APOBEC3G expression in cells are also encompassed by the present invention.

For example, combinatorial library methods known in the art, including: biological libraries; spatially addressable parallel solid phase or solution phase libraries; synthetic Ibrary methods requiring deconvolution; the ‘one-bead one-compound’ library method; and synthetic library methods using affinity chromatography selection may be used in order to identify modulators of APOBEC3G biological activity. The biological library approach is limited to peptide libraries, while the other four approaches are applicable to peptide, non-peptide oligomer or small molecule libraries of compounds (Lam, Anticancer Drug Des. 12: 145, 1997). Examples of methods for the synthesis of molecular Ibraries can be found in the art, for example in: DeWitt et al. (1993) Proc. Natl. Acad. Sci. USA. 90:6909; Erb et al. (1994) Proc. Natl. Acad. Sci. USA 91:11422; Zuckermann et al. (1994), J. Med. Chem. 37:2678; Cho et al. (1993) Science 261 :1303; Carrell et al. (1994) Angew. Chem, Int. Ed Engl. 33:2059; and ibid 2061; and in Gallop et al. (1994). Med Chem. 37:1233. Libraries of compounds may be presented in solution (e.g. Houghten (1992) Biotechniques 13:412-421) or on beads (Lam (1991) Nature 354:82-84), chips (Fodor (1993) Nature 364:555-556), bacteria or spores (Ladner U.S. Pat. No. 5,223,409), plasmids (Cull et a/.(1992) Proc Natl Acad Sci USA 89:1865-1869) or on phage (Scott and Smith (1990); Science 249:386-390). Examples of methods for the synthesis of molecular libraries can be found in the art, for example in: DeWitt et al. (1993) supra; Erb et al. (1994) supra; Zuckermann et al. (1994) supra; Cho et al. (1993) supra; Carrell et al. (1994) supra, or luciferase, and the enzymatic label detected by determination of conversion of an appropriate substrate to product. The choice of a particular combinatorial library depends on the specific APOBEC3G activity that needs to be modulated.

All methods and assays of the present invention may be developed for low-throughput, high-throughput, or ultra-high throughput screening formats. Of course, methods and assays of the present invention are amenable to automation. Automation and low-throughput, high-throughput, or ultra-high throughput screening formats is possible for the screening of agents which modulates the level and/or activity of APOBEC3G.

Generally, high throughput screens for APOBEC3G modulators i.e. viral inhibitors, candidate or test compounds or agents (e.g., peptides, peptidomimetics, small molecules, or other drugs) may be based on assays which measure a biological activity of APOBEC3G (or of Vif). The invention therefore provides a method (also referred to herein as a “screening assay”) for identifying modulators, which have an inhibitory effect on, for example, a Vif biological activity, or which bind to or interact with a Vif and/or Gag, or which have an inhibitory effect on, for example, the production of HIV.

The assays described above may be used as initial or primary screens to detect promising lead compounds for further development. Often, lead compounds will be further assessed in additional, different screens. Therefore, this invention also includes secondary APOBEC3G screens which may involve assays utilizing mammalian cell lines expressing APOBEC3G, and/or Vif, and/or Gag.

Tertiary screens may involve the study of the identified modulators in the appropriate rat and mouse models (e.g. MAIDS). Accordingly, it is within the scope of this invention to further use an agent identified as described herein in an appropriate animal model. For example, a test compound identified as described herein (e.g., a Vif inhibiting agent,) can be tested in an animal model for a homologous targeted virus to determine the efficacy, toxicity, or side effects of treatment with such an agent. Furthermore, this invention pertains to uses of novel agents identified by the above-described screening assays for treatment of viral diseases.

Other objects, advantages and features of the present invention will become more apparent upon reading of the following non-restrictive description of preferred embodiments thereof, given by way of example only with reference to the accompanying drawings.

### BRIEF DESCRIPTION OF THE DRAWINGS

In the appended drawings:

FIG. 1 shows the incorporation of APOBEC3G into viruses or Gag viral-like particles (VLPs). 293T cells were cotransfected with APOBEC3G expression vector and different plasmids containing wild-type or mutant HIV-1 proviral DNA. The plasmids used are listed along the top of each panel, and described in the text. 48 hours post-transfection, cells, viruses, or Gag VLPs produced by the cells were purified, lysed in RIPA buffer, and cellular and viral proteins were analyzed by Western blots. A. Western blots of cell lysate were probed with anti-HA (top panel), anti-β-actin (middle panel), or anti-Vif (bottom panel). B. Western blots of viral or Gag VLP lysates were probed with either anti-HA (upper panel) or anti-CA (lower panel). C. 293T cells were transfected with BH10.P-Vif− or hGag. Total cellular RNA and viral RNA were extracted, and HIV-1 viral RNA in each samples were determined by dot blot hybridization, as described in Example 1. The bar graphs represent relative amount of HIV-1 viral RNA in cell lysates (upper panel) and viral lysates (lower panel), and the results are normalized to β-actin or Gag, respectively.

FIG. 2 shows the interaction of APOBEC3G with wild-type or mutant Gag in the cell. 293T cells were cotransfected with APOBEC3G expression vector and different plasmids coding for wild-type or mutant Gag proteins. Interaction between Gag and APOBEC3G was measured by the ability to co-immunoprecipitate these molecules from cell lysate with anti-HA. Panel A graphically represents the wild-type and mutant Gag variants tested (the sequences of wild type hGag are shown in SEQ ID NOs: 22 and 23). The top drawing shows the wild-type Gag domains, with numbers representing the amino acid positions. MA, matrix domain; CA, capsid domain; NC, nucleocapsid; p6, p6 domain. B. Western blots of cell lysates of transfected cells were probed with anti-CA (top) or anti-HA (bottom). C. Western blots of anti-HA immunoprecipitates from cell lysates were probed with anti-CA (top) or anti-HA (bottom). D. 293T cells were cotransfected with BH10.P-.Vif− and APOBEC3G, and the cell lysates were subjected to RNase or DNase treatment, followed by immunoprecipitation with either anti-integrase (IN) or anti-HA, respectively. The immunoprecipitates were analyzed by Western blotting, using anti-CA to detect the presence of Gag in the immunoprecipitate.

FIG. 3 shows the ability of APOBEC3G to be incorporated into wild-type or mutant HIV-1. 293T cells were cotransfected with APOBEC3G expression vector and different plasmids containing wild-type or mutant HIV-1 proviral DNA. The plasmids used are listed along the top of each panel, and described in Example 1. A. Western blots of cell lysates were probed with either anti-HA (upper), anti-CA (middle), or anti-p-actin (bottom) B. Western blots of cell lysates of Gag VLPs produced from transfected cells were probed with either anti-HA (upper) or anti-CA (bottom).

FIG. 4 shows the ability of mutant APOBEC3G to be incorporated into Gag VLPs. Plasmids coding for N- and C-terminal APOBEC3G deletion mutants were cotransfected into 293T cells with the plasmid coding for hGag. The sequences of APOBEC3G (hAag) are shown in SEQ ID NOs: 21 and 22. A. Graphic representation of the wild-type and mutant APOBEC3G variants tested. The filled rectangles represent the two catalytic sites in APOBEC3G, and the numbers represent the amino acid positions. B. Western blots of cell lysates probed, respectively, with anti-HA (top) and anti-β-actin (bottom). C. Western blots of lysates of Gag VLPs produced from these cells, probed, respectively, with anti-HA (top) and anti-CA (bottom). The APOBEC3G: β-actin and APOBEC3G:Gag ratios are listed at the bottom of panels B and C, respectively, and are normalized to the ratio obtained for wild-type APOBEC3G.

FIG. 5 shows the distribution of APOBEC3G between cytoplasm and membrane. 2 μg APOBEC3G expression vector were transfected into 293T cells, or cotransfected with 2 μg of plasmids coding for wild-type or mutant hGag. Cells were lysed hypotonically in TE buffer, and the post-nuclear supernatant was resolved by the sucrose floatation assay into membrane-bound (I) and membrane-free (B) protein, as described in Example 1. The left side of panels A to E show Western blots of gradient fractions probed with anti-HA, while the right side of each panel presents these blots, as well as blots probed with anti-CA, graphically, showing the percentage of analyzed protein in each gradient fraction. [symbol] and [symbol] represent APOBEC3G and Gag, respectively. A. Cells are transfected with the plasmid coding for APOBEC3G alone. B-E. Cells are cotransfected with the plasmid coding for APOBEC3G and plasmid(s) coding for B. hGag, C. hGag, and Vif, D. the mutant Gag ZWt-p6.Vif−, and E. the Δ1-132 hGag. “I” and “B” at the top of panel represent interface and bottom fraction in the discontinuous sucrose gradient respectively.

FIG. 6 shows that the incorporation of APOBEC3G into Gag VLPs is proportional to its cellular expression. 293T cell were cotransfected with 2 μg hGag and various amount of plasmid coding APOBEC3G. Western blots of cell lysate or Gag VLP lysates probed for APOBEC3G with anti-HA are shown in upper and lower blot, respectively. Bands in Western blots were quantitated, and the right panel plots the relative intensities of APOBEC3G expressed in the cell vs APOBEC3G incorporated into Gag VLPs.

FIG. 7 shows the effect of Vif upon both the cellular expression of APOBEC3G and its incorporation into HIV-1. 293T cells were transfected with plasmids containing either wild-type (BH10) or Vif-negative (BH10Vif−) viral DNA, or cotransfected with these plasmids plus either plasmid alone (pcDNA3.1) or this plasmid containing APOBEC3G DNA. The plasmids used are listed along the top of each panel, and described in the text. 48 hours post-transfection, cells or viruses produced by the cells, were lysed in RIPA buffer, and cellular and viral proteins were analyzed by Western blots. A. Western blots of cell lysates, containing similar amounts of β-actin (bottom panel) were probed, from top panel down, respectively, with anti-Vif, anti-HA, anti-CA, and anti-P actin. B. Western blots of viral lysates, containing similar amounts of CAp24 (bottom panel), were probed with either anti-HA (upper panel) or anti-CA (lower panel).

FIG. 8 shows the real-time PCR quantitation of newly synthesized HIV-1 DNA. DNA was extracted at different times post-infection from SupT1 cells infected with the four viral types: BH10,±hA3G; BH10Vif−, ±hA3G. Early (R-U5) and late (U5-gag) minus strand cDNA production was monitored by real-time PCR, as described in Methods. A. The arrows indicate the PCR primers used to detect early (U5a-R) and late (gag-U5b) minus strand DNA. B,C Production of viral early (B) and late (C) DNA in SupT1 cells infected with one of the four viral types. Data were normalized to DNA production for BH10 in the absence of hA3G. a, BH10, pcDNA3.1; b, BH10Vif−, pcDNA3.1; c, BH10, hA3G; d, BH10Vif−, hA3G.

FIG. 9 shows the effect of human APOBEC3G (hA3G) upon tRNA^{Lys3} annealing to viral RNA and initiation of reverse transcription in wild-type and Vif-negative HIV-1. Total viral RNA was used in an in vitro reverse transcriptase reaction as the source of primer tRNA^{Lys3} annealed to genomic RNA in vivo. A. Cartoon showing tRNA^{Lys3} annealing and initiation of reverse transcription. The cartoon shows the tRNA^{Lys3}/genomic RNA annealing complex. This shows the annealing of the terminal 3′ 18 nucleotides of tRNA^{Lys3} to the primer binding site (PBS) on the viral RNA genome, which contains 18 complementary nucleotides. The first 6 deoxyribonucleotides incorporated (CTGCTA) during initiation of reverse transcription are underlined. B. C. D. 1D PAGE of radioactive reverse transcription products. The in vitro reverse transcription reaction, containing exogenous HIV-1 RT, uses either purified tRNA^{Lys3} heat-annealed in vitro to synthetic viral genomic RNA (lane 1), or viral RNA extracted from the four types of virions as the source of primer tRNA^{Lys3}/viral RNA template. In addition, the reaction mixtures contain either 5 μM α-³²P-GTP, 200 μM CTP and TTP, and 200 μM ddATP (B), 5 μM α-³²P-CTP (C) or 5 μM α-³²P-GTP (D). Quantitation of RT products by phosphor-imaging is shown at the right side of panels. a, BH10, pcDNA3.1; b, BH10Vif−, pcDNA3.1; c, BH10, hA3G; d, BH10Vif−, hA3G.

FIG. 10 shows the effect of increasing amounts of hA3G upon tRNA^{Lys3} annealing to viral RNA in wild-type and Vif-negative HIV-1. A, B. Western blots of cell (A) or viral (B) lysates. A, blots probed, respectively, with anti-HA and anti-β-actin. B, blots probed, respectively with anti-HA and anti-CA. C. 1D PAGE of radioactive reverse transcription products (tRNA^{Lys3} extended 6 bases, as described for FIG. 9B) using viral RNA extracted from the four types of virions as the source of primer tRNA^{Lys3}/viral RNA. Quantitation of RT products by phosphorimaging is shown at the bottom of panel C.

FIG. 11 shows viral early and late DNA production, and tRNA^{Lys3} annealing in SupT1 cells infected with BH10Vif− containing either wild-type or mutant hA3G. SupT1 cells were infected with BH10Vif− containing either no hA3G (a), wild-type hA3G (b), or mutant hA3G (c-f). A. Graphic representation of the wild-type and mutant APOBEC3G variants tested: a: no hA3G; b: wild-type hA3G; c: hA3G1O5-384; d: hA3G157-384; e: hA3G1-156; f: hA3G104-246. The filled rectangles represent the two catalytic sites (zinc coordination units) in hA3G, and the numbers represent the amino acid positions. B, C. Early and late viral DNA production. DNA was extracted at different times post-infection from SupT1 cells infected with the different viruses. Early (R-U5) and late (U5-gag) minus strand cDNA production was monitored by real-time PCR, as described in Methods, using the same PCR primers as shown in FIG. 8A. Production of viral early DNA (B) and late DNA (C) in SupT1 cells infected with the different viruses is normalized to DNA production for BH10Vif− in the absence of hA3G (a). D. tRNA^{Lys3} annealing to viral RNA in BH10Vif− containing wild-type or mutant hA3G. Total viral RNA was extracted from BH10Vif− containing either no hA3G (a), wild-type hA3G (b), or mutant hA3G (c-f). The 6-base extended tRNA^{Lys3}synthesized in an in vitro reverse transcription reaction, using total viral RNA as the source of primer tRNA^{Lys3}/viral RNA template, is as described in the legend for FIG. 9B. tRNA^{Lys3} extension is normalized to that obtained for BH10Vif− containing no hA3G.

FIG. 12 shows the ability of mutant hA3G to be degraded by Vif and bind to Vif. Plasmids coding for HA-tagged N- and C-terminal hA3G deletion mutants were transfected into 293T alone, or cotransfected with the plasmid coding for Vif. A. Graphic representation of the wild-type and mutant hA3G variants tested. The filled rectangles represent the two catalytic sites in hA3G, and the numbers represent the amino acid positions. B. Western blots of lysates of transfected cells probed, respectively, with anti-HA (top) and anti-β-actin (bottom). C. Western blots of cell lysates (top) or anti-HA immunoprecipitates (bottom) from cell lysates probed with anti-Vif.

FIG. 13 shows the ability of amino acids 104-245 of hA3G to be degraded by Vif . Plasmids coding for wild-type hA3G or hA3G 104-245 were transfected into 293T alone, or cotransfected with the plasmid coding for Vif in the presence or absence of proteasome inhibitor MG132. A. Graphic representation of the wild-type and mutant hA3G variants tested. The filled rectangles represent the two catalytic sites in APOBEC3G, and the numbers represent the amino acid positions. B. Western blots of lysates of transfected cells probed, respectively, with anti-HA (top) and anti-β-actin (bottom).

FIG. 14 shows the effect of hA3G1-156 or hA3G 157-384 upon Vif-mediated degradation of full length hA3G. 293T cell were transfected with plasmids coding for Vif (1 μg) and full-length hA3G (1 μg), and increasing amount of plasmids expressing hA3G 1-156 (A) or hA3G 157-384 (B) (0.5, 1, and 2 μg, repectively). Western blots of lysates of transfected cells probed with anti-HA.

FIG. 15 shows the effect of hA3G 1-156 or hA3G 157-384 on the interaction between Vif and full length hA3G. 293T cell were transfected with plasmids coding for Vif, fulllength hA3G, and Flag-tagged hA3G 1-156 or hA3G 157-384. Western blots of lysates of transfected cells probed, respectively, with anti-HA (top), anti-Flag (upper middle) and anti-Vif (lower middle). The bottom panel shows western blots of anti-HA immunoprecipitates from cell lysates probed with anti-Vif.

FIG. 16 shows that the expression of hA3G 1-156 or hA3G 157-384 inhibits HIV-1 replication in H9 cells. 293T cells were transfected with wild-type HIV-1 BH10. 48 hours posttransfection, the virus-containing supernatants were assayed for viral CAp24, and cellfree supernatants containing 5 ng viral CAp24 were used to infect 3×10⁶ H9 cells stably expressing hA3G 1-156, hA3G 157-384, and empty vector pcDNA3.1 as control, respectively, in 2 ml of media. (time 0). Every three days, extracellular viral capsid (CAp24) was measured by ELISA, and plotted on a linear scale.

FIG. 17 shows an alignment of the amino acid sequences of APOBEC3G from different species: humans, chimpanzees (CPZ), African green monkey (AGM), Rhesus macaque (MAC) and mouse.

FIG. 18 shows an alignment of the amino acid sequences of Gag from different viral strains: HIV-1, HIV-2, SIV and MuLV.

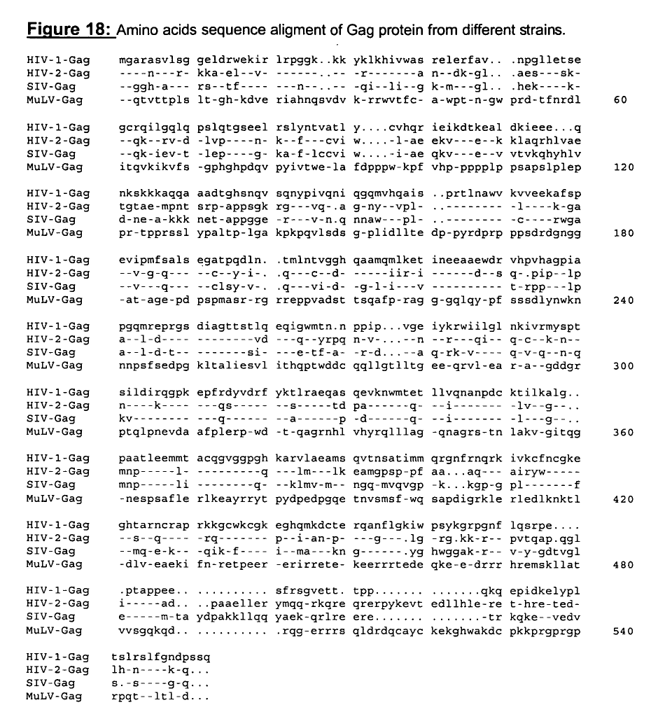

### DESCRIPTION OF ILLUSTRATIVE EMBODIMENTS

Thus, APOBEC3G, a member of an RNA/DNA cytidine deaminase superfamily, has been identified as a cellular inhibitor retroviruses, including lentiviruses and more specifically MLV, SIV, HCV, MBV, EIAV and more notably of HIV-1 infectivity, possibly through the dC to dU deamination of the first minus strand cDNA synthesized during reverse transcription. Virions incorporate APOBEC3G during viral assembly in non-permissive cells, and this incorporation is inhibited by the viral protein Vif. The mechanism of APOBEC3G incorporation into HIV-1 was examined herein. In summary it is shown that in the absence of Vif, cytoplasmic APOBEC3G becomes membrane-bound in cells expressing HIV-1 Gag, and its incorporation into Gag VLPs is proportional to the amount of APOBEC3G expressed in the cell. The expression of Vif, or mutant Gag unable to bind to the membrane, prevents the APOBEC3G association with the membrane. HIV-1 Gag alone among viral proteins is sufficient for packaging of APOBEC3G into Gag VLPs, and this incorporation requires the presence of Gag nucleocapsid. The presence of amino acids 104-156 in APOBEC3G, located in the linker region between two zinc coordination motifs, is also required for its incorporation into Gag VLPs. Evidence against an RNA bridge facilitating the Gag/APOBEC3G interaction includes data indicating that: 1) the incorporation of APOBEC3G occurs independently of viral genomic RNA; 2) a Gag/APOBEC3G complex is immunoprecipitated from cell lysate after RNase treatment; and 3) the zinc coordination motif, rather than the regions flanking this motif, have been implicated in RNA binding in another family member, APOBEC1.

The human cytidine deaminase APOBEC3G (hA3G) is expressed in non-permissive human cells, such as primary T lymphocytes, macrophages, and some T-cell lines, including H9¹⁻¹². Vif-negative HIV-1 produced in human cells containing hA3G have a severely reduced ability to produce viral DNA in newly-infected cells^{16,67,68}. It has been postulated that this reduced DNA content is due to viral DNA degradation induced by hA3G-facilitated deamination of the newly-synthesized DNA¹⁶⁻¹⁹. However, this hypothesis has yet to be proven, and recent reports have shown anti-viral activity of hA3G against both HIV-1¹⁹ and hepatitis B virus⁷⁰, independently of its deaminase activity. In HIV-1, early DNA synthesis is initiated from a cellular tRNA, tRNA^{Lys3}, that is annealed to the viral RNA genome . It is shown here that the 55-70% reduction in early viral DNA content correlates with a similar reduction in tRNA^{Lys3} annealing to the RNA genome, and this occurs in the absence of RNA deamination. Neither tRNA^{Lys3} nor viral RNA in regions of annealing show deamination mutations. Furthermore neither N nor C terminal fragments of hA3G which lack the ability to deaminate viral DNA retain the ability to reduce early and late viral DNA synthesis, tRNA^{Lys3} annealing, and viral infectivity.

Human APOBEC3G (hA3G) prevents HIV-1 replication by preventing viral DNA production. As a counter measure, the HIV-1 protein, Vif, causes the degradation of hA3G by binding to it, and directing it to the cellular proteosome for degradation. Herein hA3G deletion mutants were used to map the region in hA3G required for its degradation by Vif to hA3G amino acid residues 105-245 of SEQ ID NO: 21, the linker region between the two zinc coordination motifs. Amino acids 105-156 of hA3G are required for Vif interaction with hA3G, but not sufficient for hA3G degradation. Amino acids 157-245 (see SEQ ID NO: 21) are further required, perhaps for binding to unknown cell factors required for hA3G degradation, and/or for targeting the hA3G/Vif complex to the proteosome. The effect of expression of hA3G fragments 1-156 and 157-384 on the ability of Vif to mediate the degradation of full length hA3G showed that both fragments inhibit Vif-mediated hA3G degradation, even though coimmunoprecipitation studies indicate that only the N-terminal fragment inhibits Vif/hA3G interaction. H9 cells naturally producing hA3G, and stable H9 cell lines expressing either hA3G 1-156 or hA3G 157-384 were established. In H9 cells expressing either hA3G 1-156 or hA3G 157-384, viral production was decreased 66% and 92%, respectively, as compared to viral production in wild-type H9 cells expressing only full-length hA3G. This supports the biological effect of these fragments on the reduction of Vif-mediated hA3G degradation. Herein, therefore it is demonstrated that hA3G-derived peptides can be used to neutralize Vif's function, resulting in the inhibition of HIV-1 replication.

The present invention is illustrated in further details by the following non-limiting examples.

### EXAMPLE 1

### Experimental Procedures

Plasmid construction—SVC21BH10.P—is a simian virus 40-based vector that contains full-length wild-type HIV-1 proviral DNA containing an inactive viral protease (D25G), and was obtained from E. Cohen, University of Montreal. SVC21BH10.FS—contains mutations at the frameshift site, (i.e., from 2082-TTTTTT-2087 to 2082-CTTCCT-2087), which prevents frameshifting during the translation of Gag protein, and generates viruses that contain Gag, but not Gag-Pol (25). ZWt-p6 encodes a full-length HIV-1 genome, in which the nucleocapsid sequence has been replaced with a yeast leucine zipper domain (26). BH10.Vif−, BH10.P-.Vif−, BH10.FS-.Vif− and ZWt-p6.Vif− were generated by introducing a stop codon right after ATG of the Vif reading frame at 5043, using a site-directed mutagenesis Kit (Stratagene) with the following pair of primers: 5′-AGA TCA TTA GGG ATT TAG GM AAC AGA TGG CAG (SEQ ID NO: 2, and 5′-CTG CCA TCT GTT TTC CTA AAT CCC TAA TGA TCT (SEQ ID NO; 3).

The human APOBEC3G cDNA was amplified from H9 mRNA by reverse transcription-PCR, using the pair of primers: 5′-GCC AGA ATT CM GGA TGA AGC CTC ACT TCA G (SEQ ID NO: 4), and 5″-TAG MG CTC GAG TCA AGC GTA ATC TGG AAC ATC GTA TGG ATA GTT TTC CTG ATT CTG GAG AAT GG (SEQ ID NO: 5). The cDNA fragment was cloned into the pcDNA3.1 V5/His A vector (Invitrogen), which expresses wild-type human APOBEC3G with a fused HA tag at the C-terminus. In order to construct mutant APOBEC3G, this cDNA was PCR-amplified and digested with EcoRI and XhoI, whose sites Were placed in each of the PCR primers. These fragments were cloned into the EcoRI and XhoI sites of the pcDNA3.1 V5/His A vector. The following primers were used: wild-type: forward primer: 5′-TM GCG GAA TTC ATG MG CCT CAC TTC AGA (SEQ ID NO: 6); reverse primer: 5′-TAG MG CTC GAG TCA AGC GTA ATC TGG AAC (SEQ ID NO: 7). Δ1-57: 5′-TAG GCG GM TTC ATG GTG TAT TCC GAA CTT MG (SEQ ID NO: 8). Δ1-104: 5′-TAA GTC GAA TTC ATG GCC ACG TTC CTG GCC GAG (SEQ ID NO: 9). Δ1-1 56: 5′-TAA GTC GAA TTC ATG TTT CAG CAC TG TGG AGC (SEQ ID NO: 10). Δ157-384: 5′-TAG MG CTC GAG TCA AGC GTA ATC TGG AAC ATC GTA TGG ATA TTC GTC ATA ATT CAT GAT (SEQ ID NO: 11). Δ246-384: 5′-TAG MG CTC GAG TCA AGC GTA ATC TGG AAC ATC GTA TGG ATA CTG GTT GCA TAG AAA GCC (SEQ ID NO: 12). Δ309-384: 5′-TAG MG CTC GAG TCA AGC GTA ATC TGG AAC ATC GTA TGG ATA GAT GCA CAG GCT CAC GTG (SEQ ID NO: 13). The resulting constructs expressing HA-tagged wild-type and mutant APOBEC3G were transfected into 293T cells.

The hGag plasmid, which encodes the HIV-1 Gag sequence, produces mRNA whose codons have been optimized for mammalian codon usage, (27). All the N- or C-terminally deleted Gag plasmids were constructed using PCR. hGag was PCR-amplified and digested with SaII and XbaI, whose sites were introduced in each of the PCR primers. These fragments were cloned into the SaII and XbaI sites of hGag. The following primers were used to construct these deletions: Wild-type: forward primer: 5′-ATA ATA GTC GAC ATG GGC GCC CGC GCC AGC GTG (SEQ ID NO: 14); reverse primer: 5′-GAC TGG TCT AGA AGG GCC TCC TTC AGC TGG (SEQ ID NO: 15). Δ1-132: 5′-GCG GCG GTC GAC ATG CCC ATC GTG CAG AAC ATC (SEQ ID NO: 16). Δ284-500: 5═-GCG GCG TCT AGA TTA CAG GAT GCT GGT GGG GCT (SEQ ID NO: 17). Δ377-500: 5′-GCG GCG TCT AGA TTA CAT GAT GGT GGC GCT GTT (SEQ ID NO: 18). Δ433-500: 5′-GCG GCG TCT AGA TTA AAA ATT AGC CTG TCG CTC (SEQ ID NO: 19).

Cells, transfections and viruses purification—HEK-293T cells were grown in complete DMEM plus 10% fetal calf serum (FCS), 100 Units of penicillin and 100 μg of streptomycin per ml. For the production of viruses, HEK-293T cells were transfected using Lipofectamine™ 2000 (Invitrogen, Carlsbad, Calif.) according to the manufacturer's instructions. Supernatant was collected 48 hours post-transfection. Viruses were pelleted from culture medium by centrifugation in a Beckman Ti45™ rotor at 35,000 rpm for 1 hour. The viral pellets were then purified by centrifugation in a Beckman SW41™ rotor at 26,500 rpm for 1 hour through 15% sucrose onto a 65% sucrose cushion. The band of purified virus was removed and pelleted in 1× TNE in a Beckman Ti45™ rotor at 40,000 rpm for 1 hour. Viral RNA purification and quantitation of viral RNA and tRNA^{Lys3} by dot blot hybridization with specific DNA probes to viral RNA and tRNA^{Lys3} were as previously described⁵⁶.

Viral RNA isolation and quantification—Total cellular and viral RNA was extracted using guanidinium isothiocynate, and the relative amount of HIV-1 viral RNA was quantified by dot blot hybridization, as previously described (28). Variable known amounts of BH10 plasmid were used as a standard, and each sample of total cellular or viral RNA was blotted onto Hybond N+™ nylon membranes (Amersham Pharmacia), and was probed with a 5′³²P-end-labelled 30-mer DNA probe specific for the sequence from nt 2211 to nt 2240 of the HIV-1 genome. Experiments were done in triplicate. The amounts of HIV-1 viral RNA per sample were analyzed using phosphorimaging (BioRad)™, and the relative amount of viral RNA in cell lysates and virus preparations was determined.

Protein Analysis—Cellular and viral proteins were extracted with RIPA buffer (10 mM Tris, pH 7.4, 100 mM NaCl, 1% sodium deoxycholate, 0.1% SDS, 1% NP40™, 2 mg/ml aprotinin, 2 mg/ml leupeptin, 1 mg/ml pepstatin A, 100 mg/ml PMSF). The cell and viral lysates were analyzed by SDS PAGE (10% acrylamide), followed by blotting onto nitrocellulose membranes (Amersham Pharmacia). Western blots were probed with monoclonal antibodies that are specifically reactive with HIV-1 capsid (Zepto Metrocs Inc.), HA (Santa Cruz Biotechnology Inc.), and β-actin (Sigma), or with Vif-specific polyclonal antiserum #2221 (NIH AIDS Research and Reference Reagent Program). Detection of proteins was performed by enhanced chemiluminescence (NEN Life Sciences Products), using as secondary antibodies anti-mouse (for capsid and β-actin) and anti-rabbit (for HA and Vif), both obtained from Amersham Life Sciences. Bands in Western blots were quantitated using UN-SCAN-IT™ gel automated digitizing system.

Immunoprecipitation assay—293T cells from 100 mm plates were collected 48 hours post transfection, and lysed in 500 μl TNT buffer (20 mM Tris-HCl pH 7.5, 200 mM NaCl, 1% Triton X-100). Insoluble material was pelleted at 1800×g for 30 minutes. The supernatant was used as the source of immunoprecipitated Gag/APOBEC3G complexes. Equal amounts of protein were incubated with 30 μl HA-specific antibody for 16 hours at 4° C., followed by the addition of protein A-Sepharose (Pharmacia) for two hours. For a Western blot of different cell lysates, 500 μg of lysate protein was used for immunoprecipitation from each lysate, while for different nuclease experiments on the same lysate sample, approximately 200 μg of lysate protein was used for immunoprecipitation. Lysate protein was determined by the BioRad™ assay. The immunoprecipitate was then washed three times with TNT buffer and twice with phosphate-buffered saline (PBS). After the final supernatant was removed, 30 μl of 2× sample buffer (120 mM Tris HCl, pH 6.8, 20% glycerol, 4% SDS, 2% β-mercaptoethanol, and 0.02% bromphenol blue) was added, and the precipitate vas then boiled for 5 minutes to release the precipitated proteins. After microcentrifugation, the resulting supernatant was analyzed using Western blots. In the DNase and RNase treatment assay, the cell lysates were pre-treated with 20 μg DNase or RNase before the immunoprecipitation, as previously described (29).

Subcellular fractionation and sucrose floatation assay—Cells were lysed 48 hours post-transfection at 4° C. by dounce homogenization in 1.0 ml hypotonic TE buffer (20 mM Tris-HCl, pH 7.4, 1 mM EDTA, 0.01% β-mercaptoethanol), supplemented with protease inhibitors cocktail (“Complete”™, Boehringer Manheim). The cell homogenate was then centrifuged at 1500×g for 30 minutes to remove nuclei and unbroken cells. 0.5 ml of the resulting supernatant (S1) was mixed into 3 ml of final 73% sucrose. 7 ml of 65% sucrose in TNE (20 mM Tris pH 7.8, 100 mM NaCl, 1 mM EDTA) were layered on top of the 73% sucrose, and 1.5 ml of 10% sucrose was layered on top of the 65% sucrose. The gradients were then centrifuged at 100,000×g in a Beckman SW55 Ti™ rotor overnight at 4° C. Two ml fractions were collected, diluted with 10 ml TNT, and each fraction was centrifuged at 100,000×g at 4° C. for 1 hour. The pellets from each fraction were dissolved in SDS sample buffer, and analyzed by SDS-PAGE and Western blotting.

Measuring tRNA^{Lys3} annealing to viral RNA and the initiation of reverse transcription.—Total viral RNA isolated from virus produced in transfected 293T cells was used as the source of a primer tRNA-template complex in an in vitro reverse transcription reaction, and used to measure both the amount of extendable tRNA^{Lys3} annealed to viral RNA, and the ability of this annealed tRNA to initiate reverse transcription, as previously described (1, 2, 56). Briefly, total virus RNA was incubated at 37° C. in 20 ml of RT buffer (50 mM Tris-HCl[pH7.5], 60 mM KCl, 3 mM MgCl₂, 10 mM dithiothreitol) containing 50 ng of purified HIV RT, 10 U of RNasin™, and various radioactive α-³²P-deoxynucleotide triphosphates (dNTPs). The extension product was ethanol precipitated, resuspended, and analyzed on 6% polyacrylamide-7M urea-1× tris-borate-EDTA. Initiation from unextended tRNA^{Lys3} was measured in the presence of the first base incorporated, dCTP, while initiation from 2 base-extended tRNA^{Lys3} (tRNA^{Lys3}-CT) was measured in the present of the 3rd base incorporated, dGTP. To measure total tRNA^{Lys3} annealing to viral RNA (which includes both unextended and 2-base-extended forms of tRNA^{Lys3}), the reaction mixture contained 200 μM dCTP, 200 μM dTTP, 5 μCi of?α-³²P-dGTP(0.16 μM), and 50 μM ddATP. In some experiment, NCp7 was incubated with total viral RNA for 30 min at 37° C. in RT buffer, and removed by proteinase K digestion and phenol-cholroform extraction as described previously (1), followed by initiation of reverse transcription. For example 7, separate measurements of the annealing of unextended or 2 base-extended tRNA^{Lys3} were also performed in the presence of either the first base incorporated, α-³²P-dCTP, or the 3^{rd} base incorporated, α-³²P-dGTP. Reaction products were resolved using 1D 6% PAGE⁵⁶.

Nucleocapsid protein—Recombinant HIV-1 nucleocapsid protein (NCp7) composed of 55 amino acids, was expressed in bacteria as previously described. The primer/template complex was pre-incubated with with 10 pmolar NCp7 in RT buffer at 37° C. for 30 min. The NCp7 was then removed by proteinase K digestion and phenol-chloroform extraction. Reverse transcription was initiated through the addition of RT, and the reaction was incubated for 30 minutes, and then analyzed by 1D PAGE. The results indicate that the reduced initiation of reverse transcription seen in Vif-negative viruses produced from 293T cells expressing APOBEC3G is rescued 40-70% when the total viral RNA is transiently exposed b mature nucleocapsid protein. Exposure to nucleocapsid of the total viral RNA isolated from wild-type viruses produced in APOBEC3G-expressing cells has no effect upon initiation of reverse transcription.

Real-time PCR quantitation of newly synthesized HIV-1 DNA—Equal amounts of DNase-treated virions (100 ng p24) were used to infect 1×10⁶ SupT1 cells in a volume of 1.5 ml on ice. Following 1 hour incubation on ice, the infected cells were washed twice with PBS, and aliquots of 1×10⁵ infected SupT1 cells were plated into 6-well plates containing complete RPMI 1640 medium pre-warmed to 37° C., and incubated at 37° C. At different time point post-infection, aliquots of cells were collected, washed with PBS, and cellular DNA was extracted using the DNeasy™ Tissue Kit (Qiagen). Early (R-U5) and late(U5-gag) minus strand reverse transcripts were quantitated by the LightCycler™ Instrument (Roche Diagnostics GmbH) using the following primers: early RT forward (5′-TTAGACCAGATCTGAGCCTGGGAG; SEQ ID NO: 25) and early RT reverse (5′-GGGTCTGAGGGAT CTCTAGTTACC; SEQ ID NO: 26); late RT forward (5′-TGTGT GC CCGTCTGTTGT-GTGA; SEQ ID NO: 27) and late RT reverse (5′-GAGTCCTGCGTCGAGAGAG CT; SEQ ID NO: 28).

Viral RNA and tRNA^{Lys3} sequences—RT/PCR was performed upon total viral RNA using SuperScript™ One-Step RT/PCR with Platinum™ Taq (Invitrogen Life Technologies). The primers were: forward primer (469-492):5′CCAGATCTGAGCC TGGGAGCTC (SEQ ID NO: 29); reverse primer (764-789): 5′CTCCTTCTAGCCT CCGCTATC (SEQ ID NO: 30). The PCR products were inserted into the pCR4-TOPO™ vector (Invitrogen Life Technologies) and individual clones were sequenced. To sequence viral tRNA, low molecular-weight tRNA was purified from total viral RNA using AX-20 chromatography (Biotech) and 3′ polyadenylated⁷⁴. The polyA+ RNA was annealed with 5′-TTGAATTCGCATTGAGCAC CTGCTTTTTTTTTTTTTTTTTTGG-3′ (SEQ ID NO: 31), which was used to prime cDNA synthesis using superscript II™ (Gibco). The RNA template was digested with Rnase H and Rnase A. A phosphorylated, blocked anchor-oligonucleotide, 5′-pTCTTTAGTGAGGGTTMTTGCCAdd-3′ (SEQ ID NO: 32), was ligated to the 3′-terminus of cDNA using T4 RNA ligase. The purified cDNA-anchor-oligonucleotide was amplified by PCR with the forward primer 5′-TTGMTTCGCATTGAGCACCTGC-3′ (SEQ ID NO: 33) and reverse primer 5′-GGCAATTAACCCTCAC TAAAG-3′ (SEQ ID NO: 34). The PCR products were purified with agarose electrophoresis, and cloned into the pCR-2.1-TOPO vector using the TOPO™ TA cloning kit (Invitrogen). The plasmid DNA constructs were then sequenced.

Viral genomic DNA sequencing—Viral supernatants from transfected 293T cells were filtered through 0.45 mM filters and treated with DNase at 20 IU/ml for 1hour at 37° C. to prevent proviral DNA carryover. Ten ng viral p24 was used to infect 2×10⁵ Sup-T1 cells in a volume of 1.5 ml RPMI medium. After 4 hours incubation, the infected cells were washed twice with PBS, and plated into 6-well plates. Complete RPMI 1640 medium pre-warmed to 37° C. was added to the infection mixture. Cultured cells were collected 24 hours post-infection, and DNA was extracted using DNeasy™ Tissue Kit (Qiagen). PCR was performed with Platinum™ Taq polymerase (Invitrogen Life Technologies). The primers were as follows: Forward (469492) 5′-CCAGATCTGAGC CTGGGAGCTC-3′(SEQ ID NO: 35; reverse primer(764-789) 5′-CTCCTTCTAGCCTCCGCTAGTC-3′ (SEQ ID NO: 36). The PCR products were cloned into pCR4-TOPO™ vector(Invitrogen Life Technologies) and individual clones were sequenced.

### EXAMPLE 2

### Incorporation of APOBEC3G into Gag VLPs

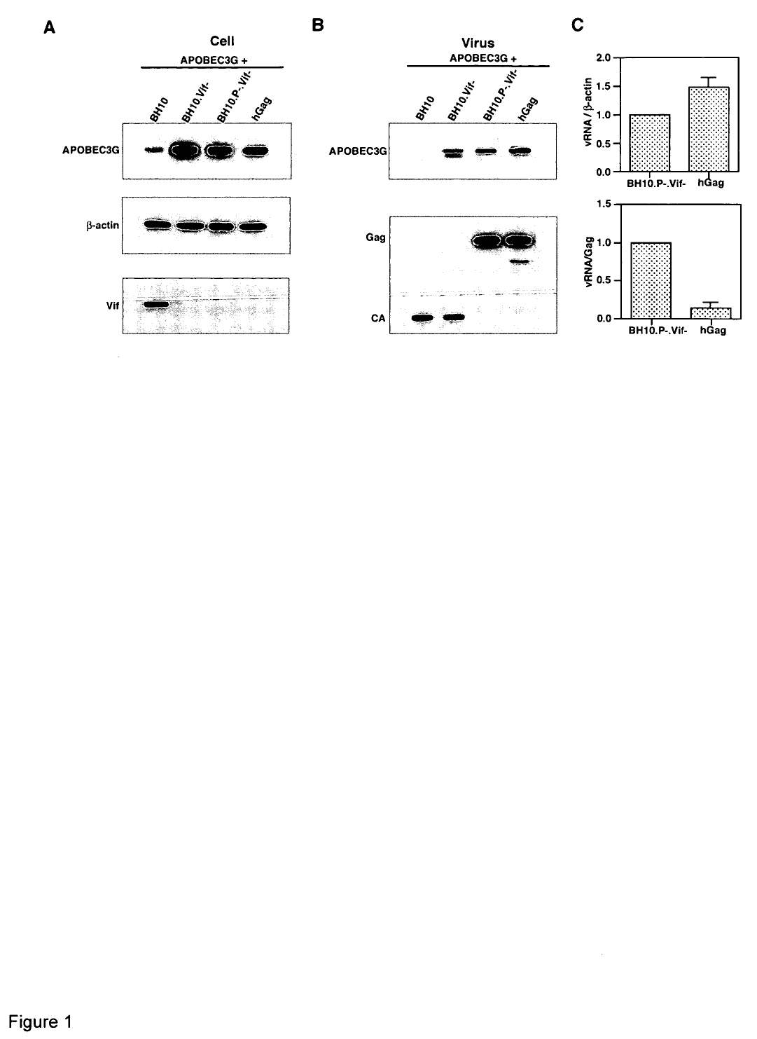

293T cells were co-transfected with a plasmid coding for human APOBEC3G containing a C-terminal HA tag, and plasmid containing wild type or mutant HIV-1 proviral DNA. BH10.Vif− and BH10.P-.Vif− both contain a stop codon immediately after the initiation ATG codon of the Vif reading frame, and BH10P-contains an inactive viral protease. hGag contains a humanized HIV-1 Gag gene (i.e., codon usage optimized for translation in mammalian cells (27)), and only wild type HIV-1 Gag and Gag VLPs are produced (25). The cell lysates of transfected cells were analyzed by Western blots (FIG. 1A), using anti-HA (top panel), anti-β-actin (middle panel) and anti-Vif (bottom panel) antibodies as probes. Vif is detected only in cells transfected with BH10. In cells producing virions or Gag VLPs lacking Vif, APOBEC3G is is strongly expressed, while in cells producing BH10, very little APOBEC3G is seen in the cytoplasm. The viruses produced from these cells were analyzed by Western blotting ( FIG. 1B), using anti-HA (top panel) and anti-CAp24 (bottom panel). While no APOBEC3G is seen in wild-type BH10, it is found in virions not expressing Vif. These results also indicate that Gag alone is sufficient among the viral proteins for facilitating APOBEC3G incorporation. These results also confirm previous observations of a diminished presence of APOBEC3G in both the cytoplasm and in virions in the presence of Vif expression, and this has been shown to be due to the Vif-induced polyubiquitination of APOBEC3G, and subsequent degradation by the proteosome (22,23,30-32)

As well as lacking coding sequences downstream of Gag, the RNA coding for hGag has the 5′ RU5 and leader sequence of the viral RNA replaced with a CMV promoter. Therefore, it is not expected that hGag VLPs will specifically package this RNA, which lacks viral packaging signals. This suggests that APOBEC3G incorporation into these particles occurs independently of viral genomic RNA packaging. To further confirm this, total RNA was extracted from cells cotransfected with APOBEC3G and either BH10.P-.Vif− or hGag, and from the virions produced from these cells. Viral mRNA in the cells and viruses were quantified by dot blot, using a ³²P-labelled DNA probe specific for the p6 coding sequence, which is present in both BH10.P-.Vif− and hGag RNA. The ratios for viral RNA: β-actin in the cytoplasm, and viral RNA:Gag in virions, is presented graphically in FIG. 1C. Although cytoplasmic expression of viral genomic RNA is strong in cells expressing hGag (top panel, FIG. 1C), the genomic RNA/Gag in hGag VLPs is reduced to approximately 15% of that found in BH10.P-.Vif−, (bottom panel, FIG. 1C). This reduced incorporation of viral RNA does not, however, affect APOBEC3G incorporation into hGag VLPs (panel B), indicating that APOBEC3G incorporation into virions occurs independently of viral RNA incorporation.

### EXAMPLE 3

### The Nucleocapsid Sequence within Gag is Required for the Viral Packaging of APOBEC3G

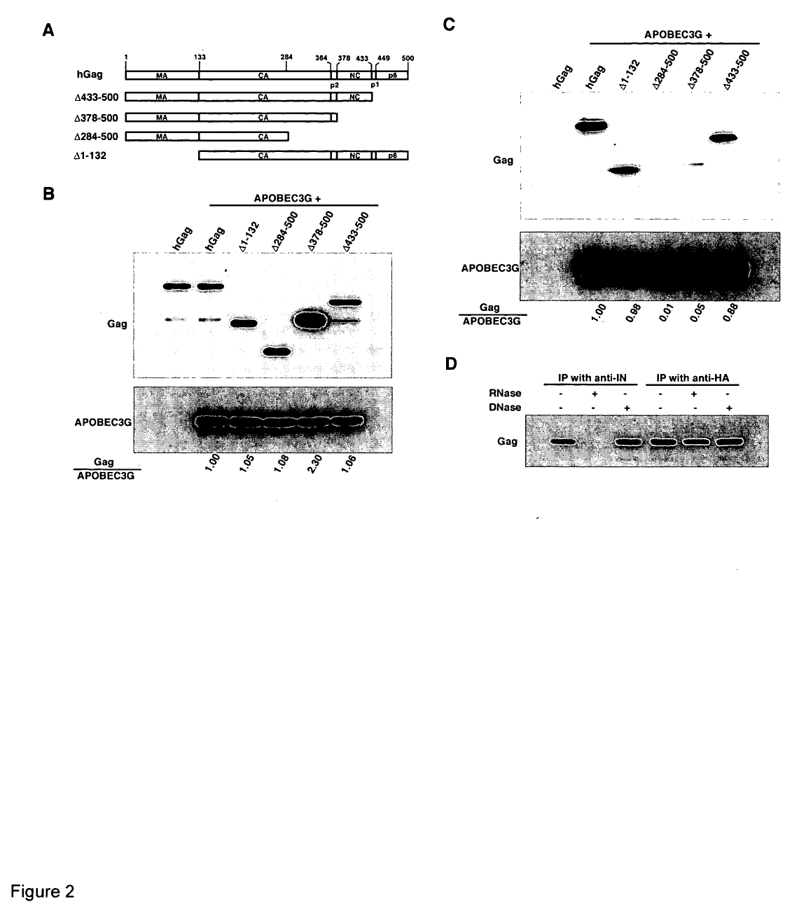

A series of Gag deletion constructs were used to identify the motif within Gag involved in the incorporation of APOBEC3G into viruses. These constructs are shown in FIG. 2A. 293T cells were cotransfected with APOBEC3G and wild-type or mutant Gag constructs, and cells were lysed in RIPA buffer. Western blots of cell lysates (FIG. 2B) were probed with anti-CA (upper panel) or anti-HA (lower panel). The first lane represents cells transfected with hGag alone. All Gag mutants were expressed at similar levels in the cytoplasm, except for the 378-500 construct. This Gag has NC, p1 and p6 deleted from the C-terminus, and is expressed 2-3 fold higher than full-length Gag.

Most of these mutant Gag molecules are impaired in their ability to form extracellular particles due to the absence of membrane- or RNA-binding regions. The interaction between APOBEC3G and mutant Gag species was therefore investigated using immunoprecipitation to detect cellular complexes. The presence of both Gag and APOBEC3G in the cell lysate was first analyzed by Western blots probed with anti-CA (FIG. 2B, upper panel), and anti-HA (FIG. 2B, lower panel). The Gag:APOBEC3G ratios, listed at the bottom of panel B, normalized to the hGag:APOBEC3G ratio, are similar for all mutant Gag species expressed, except for Δ378-500, which shows a higher expression of Gag. APOBEC3G in each cell lysate was then immunoprecipitated by anti-HA, and the presence of both Gag and APOBEC3G in the immunoprecipitate was analyzed by Western blotting, using anti-CA (FIG. 2C, upper panel), and anti-HA (FIG. 2C, lower panel). The Gag:APOBEC3G ratios, listed at the bottom of panel C, normalized to the hGag:APOBEC3G ratio, indicate no change in the association of Gag with APOBEC3G with removal of the N-terminal MA sequences (Δ1-132), and a small decrease (12%) with removal of the Gterminal p1/p6 sequences p433-500). However, a C-terminal deletion of Gag which also included NC (Δ378-500) resulted in a >95% reduction in the interaction of Gag with APOBEC3G, even though the expression of this mutant Gag is greater in the cell lysate than seen for hGag (FIG. 2B). A larger C-terminal Gag deletion (Δ284-500), in which p2 and the C terminal region of capsid (including the MHR domain) have been further removed, also prevented interaction with APOBEC3G. These data suggest that nucleocapsid sequences within Gag are responsible for the interaction between APOBEC3G and Gag. The small decrease in the Gag:APOBEC3G ratio found with removal of the p1/p6 sequences might reflect an altered conformation affecting the neighboring NC binding site in Gag.

Both Gag nucleocapsid (33) and members of the APOBEC family, including APOBEC3G (14), can bind to RNA, so that the interaction demonstrated between Gag and APOBEC3G could be mediated by an RNA bridge. However, the data in FIG. 2D suggests that an RNA bridge is not likely. 293T cells were cotransfected with BH10.P-.Vif− and APOBEC3G, and the cell lysates were subjected to RNase or DNase treatment, followed by immunoprecipitation with either anti-integrase (IN) or anti-HA, respectively. The immunoprecipitates were analyzed by Western blotting, using anti-CA to detect the presence of Gag in the immunoprecipitate. The left side of panel D shows the effects of DNase and RNase upon the immunoprecipitation of Gag with anti-IN, which reacts with GagPol. It has been reported previously that anti-IN will not immunoprecipitate Gag in the presence of RNase (29), and the results on the left side of panel D repeat those results. The right side of panel D shows a similar experiment in which APOBEC3G is immunoprecipitated with anti-HA, and the coimmunprecipitation of Gag is determined. It can be seen that exposure of the immunoprecipitate to either RNase or DNase does not affect the coimmunprecipitation of APOBEC3G with Gag. While this strongly suggests the lack of an RNA or DNA bridge between these two molecules, the possibility that a small RNA bridge may be protected from RNase digestion by the two proteins cannot be eliminated. Nevertheless the results strongly suggest that RNA is not involved in the APOBEC3G-Gag interaction.

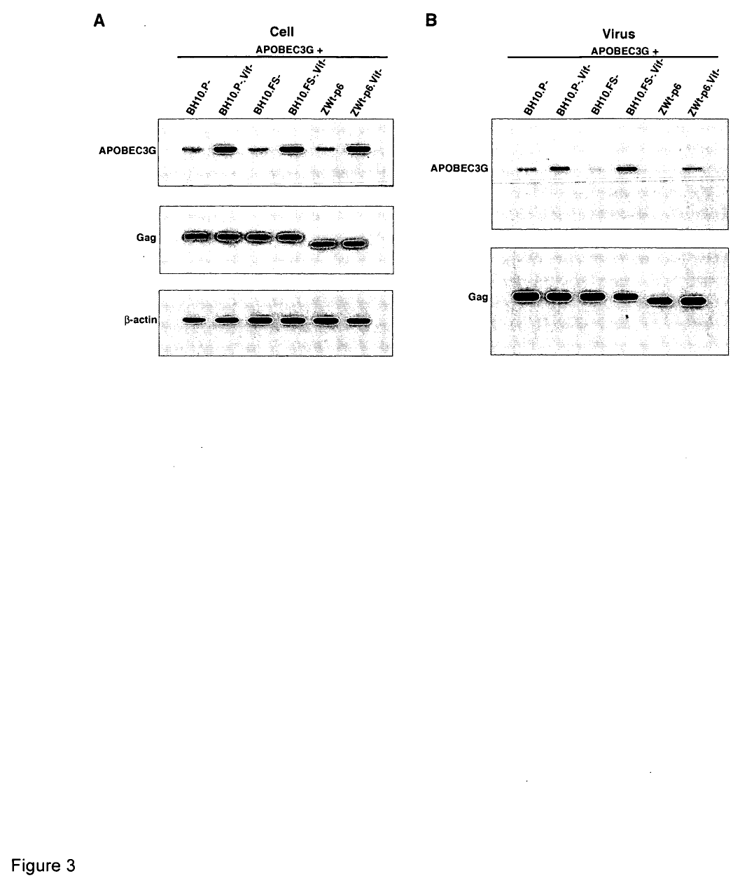

The requirement for nucleocapsid sequence is further shown in FIG. 3, in which the nucleocapsid sequence in HIV-1 has been replaced with a yeast leucine zipper domain to allow for protein/protein interactions (plasmid ZWt-p6.Vif−). It has previously been shown that the parental plasmid, ZWt-p6, can efficiently produce extracellular viruses (26). Another mutant, BH10.FS-.Vif−, in which frame shift sequence had been changed to produce only Gag, was used as a control. 293T cells were cotransfected with APOBEC3G and mutant HIV-1 plasmids, and expression of APOBEC3G in cells were analyzed by Western blots, probed with anti-HA, anti-CA, and anti-β-actin (FIG. 3A). The results show that similar amounts of APOBEC3G were efficiently produced in all the cells transfected with Vif-constructs (FIG. 3A, upper panel, lanes 2, 4 and 6), whereas cellular APOBEC3G was severely reduced if the viral constructs produced Vif (FIG. 3A, upper panel, lanes 1, 3 and 5). The absence or presence of Vif had no effect upon cellular Gag levels (FIG. 3A, middle panel). The ability of the viruses to package APOBEC3G was then assessed by Western blots of viral lysates probed with anti-CA (FIG. 3B, lower panel) or anti-HA (FIG. 3B, upper panel). The results show that BH10.FS-.Vif− can package APOBEC3G as efficiently as BH10.P-. On the other hand, the ability of ZWt-p6.Vif− to incorporate APOBEC3G is reduced 90% compared with BH10.FS-.Vif−. These data demonstrate that while the leucine zipper motif can functionally replace nucleocapsid for Gag multimerization and virus assembly, it cannot replace its ability to facilitate APOBEC3G incorporation. Thus, the incorporation of hA3G into the virion is not an indirect result of Gag multimerization but is due to an interaction with NC of Gag.

### EXAMPLE 4

### Sequences in APOBEC3G Required for its Incorporation into Gag VLPs

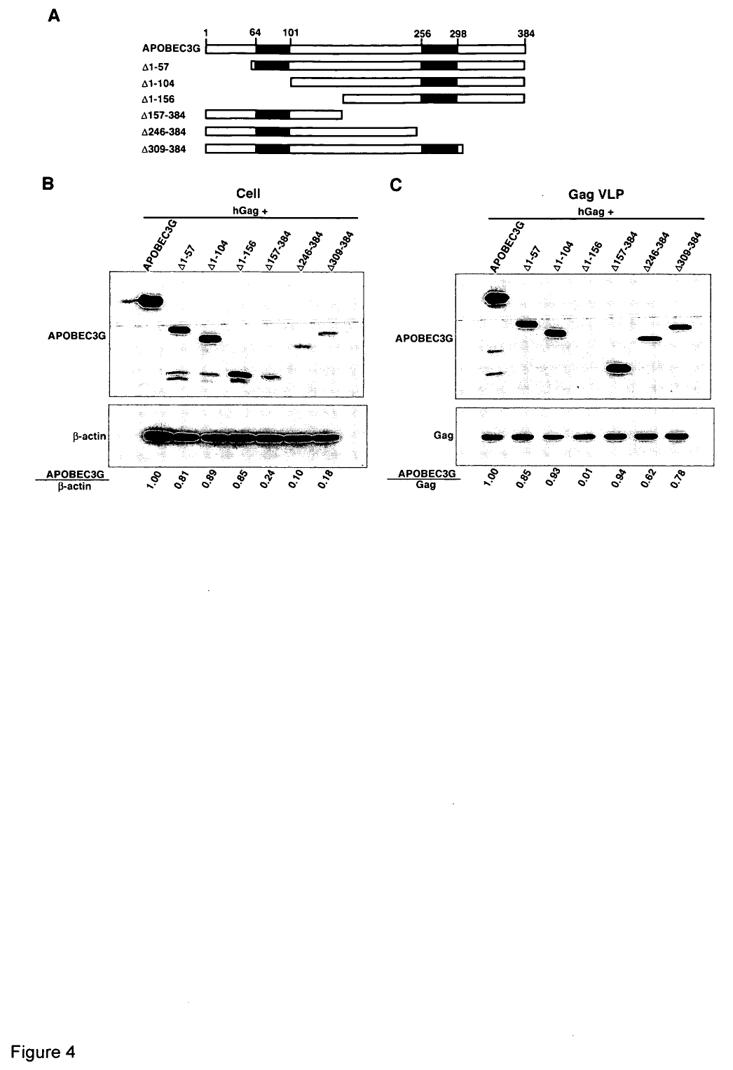

293T cells were cotransfected with hGag and a plasmid coding for wild-type or N- or C-terminal-deleted APOBEC3G tagged with HA. These constructs are shown graphically in FIG. 4A. APOBEC3G has sequence homology with APOBEC1, and contains two or one active site regions, respectively, (H-X-E-(X)₂₄₋₃₀-P-P-X-X-C: SEQ ID NO: 24) containing a zinc coordination motif (For more information on zinc coordination motif, see^{66, 75} and see below). The cytoplasmic expression and viral incorporation of the different APOBEC3G variants was determined by Western blots probed with anti-HA and anti-β-actin for cells (FIG. 4B) or anti-HA and anti-CA for viruses (FIG. 4C). The mutant APOBEC3G:β-actin ratio in the cell lysates, or APOBEC3G:Gag ratio in the viral lysates, are normalized to a ratio of 1.0 for wild-type APOBEC3G, and are listed at the bottom of each panel. As shown in FIG. 4C, deletion of the N-terminal 104 amino acids or the C-terminal 157-384 amino acids (See SEQ ID NO: 21) does not affect the ability of APOBEC3G to be packaged into Gag VLPs, whereas the deletion of the N-terminal 156 amino acids abolishes its incorporation into viruses. This result indicates that amino acids 104-156, found in the N-terminal portion of a linker sequence between the two zinc coordination motifs in APOBEC3G, are required for its incorporation into Gag VLPs.

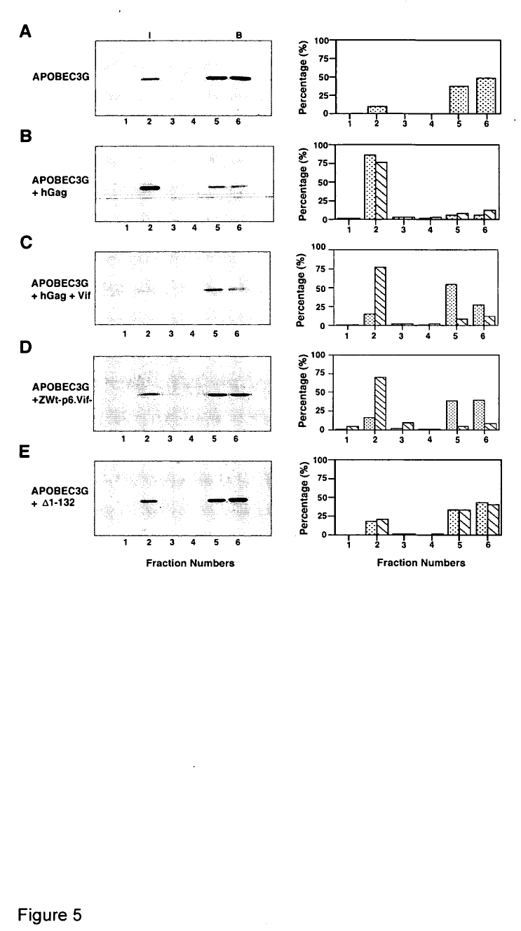

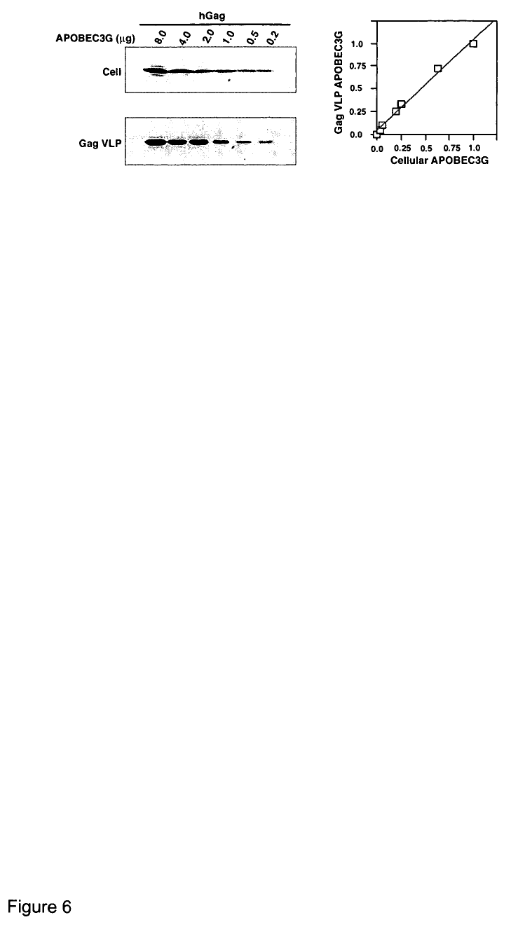

All C-terminal APOBEC3G deletions shown in FIG. 4 show reduced expression in the cell lysate (10-20% of wild-type (FIG. 4B)). This may be due to intracellular degradation since it has been reported that N-terminal fragments of APOBEC3G are inherently unstable (34). Interestingly, the viral content of these N-terminal fragments is >60% of wild type APOBEC3G, i.e., does not reflect their low cytoplasmic expression. Thus, the removal of the C-terminal regions of APOBEC3G appears to result in a significant decrease in its concentration in the total cell lysate without a similar quantitative decrease in its incorporation into Gag VLPs. This suggests that the decreased APOBEC3G pools are not the source of viral APOBEC3G. The floatation gradients of post-nuclear supernatant, as shown in FIG. 5, indicate that almost all cytoplasmic APOBEC3G interacts with Gag and moves to the membrane. However, we have recently observed that >80% of APOBEC3G is found in the nucleus (data not shown), so the decreased expression of C-terminally truncated APOBEC3G in cell lysate might involve primarily nuclear APOBEC3G, and not affect the cytoplasmic pools. The cellular source of viral APOBEC3G is currently being investigated, and might be similar to the cellular origins of viral GagPol (35) and viral LysRS (36,37). Both of these molecules are rapidly incorporated into Gag particles, and appear to come from cytoplasmic pools of newly-synthesized molecules. The alternative explanation that the C-terminally truncated APOBEC3G interacts with Gag more efficiently than wild-type Gag is not likely, since, as shown in FIG. 6, increasing concentrations of wild-type APOBEC3G in the cytoplasm interact efficiently with Gag.

### EXAMPLE 5

### Effect of Gag Expression upon the Intracellular Distribution of APOBEC3G

293T cells were transfected with the plasmid coding for APOBEC3G alone, or co-transfected with this plasmid and plasmids coding for mutant forms of hGag in the presence or absence of Vif. Transfected cells were lysed in hypotonic buffer, and, after a low-speed centrifugation to remove broken cells and nuclei, the post-nuclear supernatant was resolved on sucrose gradients into membrane-free and membrane-bound protein, as described previously (35). Gradient fractions were analyzed by Western blots, probed with anti-HA or anti-CA antibody. As shown in FIG. 5A, in the absence of Gag, >90% APOBEC3G is present near the bottom of the gradient, i. e., in the cytoplasmic fraction (lanes 5 and 6). However, in the presence of Gag (FIG. 5B), >90% of APOBEC3G is localized in the membrane-bound protein near the top of the gradient at the 10%/65% sucrose interface, reflecting a similar intracellular distribution for Gag (35). If Vif is also expressed, the APOBEC3G remains in the cytoplasm at reduced levels (FIG. 5C). When cells express both APOBEC3G and the mutant Gag species, ZWt-p6. Vif−, the majority of APOBEC3G remains in the cytoplasm even though most Gag is found at membrane (FIG. 5D). When cells are transfected with a mutant Gag that can no longer bind to membrane (Δ1-132), but that retains the ability to bind to APOBEC3G, the APOBEC3G remains in the cytoplasm (FIG. 5E). These data indicate that binding to Gag transports most cytoplasmic APOBEC3G to the membrane during viral assembly. This interaction is efficient, since when cells are cotransfected with the hGag plasmid and increasing amounts of the plasmid expressing APOBEC3G, the amount of APOBEC3G incorporation into viruses is proportional to APOBEC3G expressed in the cell (FIG. 6).

### EXAMPLE 6

### Implication of APOBEC3G Interaction with Gag

Applicants have shown that Gag alone among viral proteins is sufficient for the incorporation of APOBEC3G, and deletion analysis shows that Gag nucleocapsid and amino acids 104-156 in APOBEC3G are required for the Gag/APOBEC3G interaction. FIG. 2C shows that the cytoplasmic interaction between Gag and APOBEC3G requires NC sequences. The requirement for Gag nucleocapsid suggests a direct interaction of this Gag domain with APOBEC3G, but could also reflect a requirement for either Gag multimerization or for an RNA bridge binding the two proteins. The fact that the Gag/APOBEC3G interaction is still detected after Rnase A treatment (FIG. 2D) suggests that Gag multimerization is not required for the interaction. Furthermore, Gag multimerization is not sufficient for the incorporation of APOBEC3G into viral particles. Thus, experiments with ZWt-p6.Vif−, a virus in which the nucleocapsid sequence has been replaced with a yeast leucine zipper responsible for facilitating protein interactions, show that the resulting extracellular Gag particles produced do not incorporate APOBEC3G (FIG. 3B), i. e., the presence of NC is still required. This indicates that, while the incorporation of APOBEC3G into Gag VLPs is proportional to its expression in the cell (FIG. 6), APOBEC3G is not randomly incorporated into Gag VLPs or virions. The simple production of viral particles does not ensure a random incorporation of APOBEC3G. On the other hand, the fact that APOBEC3G is incorporated into virions with diverse Gag sequences, including HIV-1, murine leukemia virus (MLV), simian immunodeficiency virus (SIV), and equine infectious anemia virus (EIAV) (16,18) suggests that some common property of Gag NC other than sequence similarity is required. This feature could be common structural motifs, or it could be their common ability to bind RNA.

However, the data presented here, while not eliminating the existence of an RNA bridge facilitating the interaction between Gag and APOBEC3G, does not favor the prime importance of such a bridge. The RNA producing hGag does not contain viral genomic RNA packaging signals. The hGag VLPs produced, while containing only 14% as much viral genomic RNA as virions containing wild-type Gag (FIG. 1C), do efficiently package APOBEC3G (FIG. 1B). This indicates that APOBEC3G packaging occurs independently of HIV-1 viral genomic RNA, and supports an earlier finding that used a UV crosslinking assay to demonstrate that APOBEC3G bound specifically to apoB mRNA and UA rich RNA, but not to HIV-1 RNA (14). A unique role for cellular RNA in facilitating an APOBEC3G/Gag interaction is also not supported by the data. The ability to immunoprecipitate a cytoplasmic Gag/APOBEC3G complex is only slightly diminished upon prior treatment with RNase A (10-14% decrease), while the immunoprecipitation of a Gag/GagPol complex is completely inhibited by a similar RNase A treatment (FIG. 2D). However, the possibility that RNA bridging Gag and APOBEC3G is protected from RNase digestion by these proteins cannot be formally eliminated. Nevertheless, the data shown herein strongly suggest that an RNA bridge is not involved.

Although the RNA-binding region(s) within APOBEC3G are not known, they have been mapped in the related family member APOBEC1 to its single zinc coordination motif (38,39). APOBEC3G binds to zinc in vitro, and has an RNA binding capacity similar to APOBEC1 (14). Amino acids 104-156 in APOBEC3G are required for the incorporation of this molecule into Gag VLPs, yet lay outside either zinc coordination motifs. This finding does not support a major role for RNA in the Gag/APOBEC3G interaction. There also does not appear to be any local cluster of basic amino acids within amino acids 104-156 (SEQ ID NO: 1) which could contribute to the non-specific binding of RNA. Of note, little or no effect on APOBEC3G incorporation into virions was observed with the removal of either zinc coordination motif (FIG. 4C). Taken together, the data presented herein strongly suggest that RNA binding to HA3G is not a major factor in hA3G incorporation and antiviral function.

The data presented in the middle panel in FIG. 3A do not show a difference in Gag levels in Vif+ or Vif− cells expressing APOBEC3G (i. e., while the cellular expression of APOBEC3G is decreased in Vif− cells, Gag does not decrease). In fact, while the presence of Vif in non-permissive cells alters the cytoplasmic distribution of APOBEC3G, it does not alter the cytoplasmic distribution of Gag. This is shown in FIG. 5, panels A-C. APOBEC3G in the post-nuclear supernatant is found primarily in the cytoplasm of non-permissive cells (FIG. 5A). In cells also expressing Gag, almost all of APOBEC3G is carried to the membrane in the absence of Vif (FIG. 5B), but wild-type Gag does not carry APOBEC3G to the membrane in the presence of Vif (FIG. 5C). It can also be seen that the cellular distribution of Gag between membrane and cytoplasm is unaltered whether Vif is present or not. The ability of Gag to alter the cytoplasmic distribution of APOBEC3G depends upon Gag's ability to interact with either cell APOBEC3G (FIG. 5D, in which the mutant Gag species ZWt-p6.Vif− is expressed), or with the membrane (FIG. 5E, in which the Δ1-132 mutant Gag species, which lacks membrane-binding sequences, is expressed).

The data in FIGS. 3 and 5 suggest that little, if any, Gag is associated with the Vif/APOBEC3G complex. Although immunofluorescence studies showed a colocalization of Gag and Vif in the cell (40), cosedimentation studies indicated an interaction of Vif only with some early viral assembly intermediates, and the presence of Vif in mature virions remains controversial (41-48). In insect cells infected with baculovirus expressing Gag and Vif, it was estimated that there were 70 Vif molecules per 2000 Gag molecules in extracellular Gag particles, or one molecule of Vif for every 30 molecules of Gag (49). If single Gag molecules bound to Vif at this same ratio within an APOBEC3G/Vif/Gag complex destined for degradation in the proteosome, this would account for only 3.5% of Gag molecules produced, and a change in Gag distribution in the cell would not be detectable by the Western blot assay shown herein.

Alternatively, the formation of an APOBEC3G/Vif/Gag complex may be prevented by overlapping binding sites. While the ability to coimmunoprecipitate Gag and Vif from cell lysates has met with varying degrees of success (50,51), the in vitro interaction between Vif and Gag has been used to map interacting sites on these two molecules (49). These results indicate that the Vif binding sites on Gag include the C terminal of NC (including the second zinc finger), the spacer peptide sp2, and the N terminal region of p6. Since NC is involved in binding to both Vif and APOBEC3G, the latter two molecules might compete for binding to Gag. Similarly, the APOBEC3G binding sites for Vif and Gag have been estimated to include amino acids 54-124 for Vif (34), and amino acids 104-156 for Gag, as reported herein. The lack of formation of a Gag/Vif/APOBEC3G complex could therefore also be due to competitive binding between Gag and Vif for sites on APOBEC3G, or to conformational restraints preventing both molecules binding to APOBEC3G.

Most cytidine deaminases act as homodimers or homotetramers (52,53). It has been reported for APOBEC1 that small N-(10 amino acids) or C-(10 amino acids) terminal deletions reduce RNA editing, RNA binding, and homodimerization activities (53). Similarly, it has been reported for APOBEC3G that N- and C-terminal deletions which do not eliminate either active site, still destroy enzyme activity, and that this is due to inhibition of APOBEC3G dimerization (54). It is shown herein that larger N- and C-terminal deletions of APOBEC3G can still be packaged into HIV-1 (FIG. 4). This suggests that neither APOBEC3G dimerization, nor its binding to RNA is required for this packaging process.

It is not clear if the deoxycytidine deaminase activity of APOBEC3G is the sole determinant in inhibiting HIV-1 replication. For example, while two reports have indicated that mutations in either active site result in similar losses of both deoxycytidine deaminase activity and anti-viral activity (16,17), a more recent paper reports that mutations in either active site inhibit deoxycytidine deaminase activity to different extents, but have the same anti-viral activity (54). This latter observation implies that deoxycytidine deaminase activity of APOBEC3G may not be the sole determinant for anti-viral activity. It is possible that the interaction of APOBEC3G with nucleocapsid might result in the inhibition of viral functions associated with nucleocapsid. For example, Gag nucleocapsid sequences facilitate tRNA^{Lys3} annealing to viral genomic RNA (55), which could explain the observation that deproteinized viral RNA (which contains primer tRNA^{Lys3} annealed to viral genomic RNA) extracted from Vif-negative HIV-1 produced in non-permissive cells shows a decreased ability to support reverse transcription in vitro compared to the same RNA extracted from similar virions produced in permissive cells (8). Alternatively, this observation might reflect the presence in non-permissive cells of other anti-HIV-1 factors yet to be discovered.

### EXAMPLE 7

### Human APOBEC3G Inhibits both Viral DNA Replication and Primer tRNA^{Lys3} Annealing in HIV-1 Independently of its Cytidine Deaminase Activity

The initiation of reverse transcription in HIV-1 requires tRNA^{Lys3} as a primer, and this tRNA is packaged into the virus during its assembly. tRNA^{Lys3} is annealed to a region near the 5′ end of the viral RNA termed the primer binding site (PBS), and used to prime the reverse transcriptase-catalyzed synthesis of minus strand cDNA, the first step in reverse transcription. It has been reported previously that Vif-negative virions produced from H9 cells, a non-permissive cell line, have approximately 50% reduced annealing of primer tRNA^{Lys3}, and >90% reduction in initiation of reverse transcription, compared to Vif-positive virions (8). The implication of these results is that even if some tRNA^{Lys3} is annealed to the viral genome, it is not placed properly to initiate reverse transcription. A similar situation has also been reported when comparing tRNA^{Lys3} annealing to the viral RNA genome in wild-type vs protease-negative HIV-1 (56). In that report, annealing and initiation of reverse transcription in the protease-negative virus were rescued through the transient addition of mature HIV-1 nucleocapsid (NCp7) to the viral RNA/primer tRNA^{Lys3} template used to measure these parameters. Both Gag (55, 56, 57) and mature nucleocapsid (NC) (58, 59) have been shown to facilitate the annealing of tRNA^{Lys3} to viral RNA, in vitro and in vivo. The data presented hereinbelow indicate that APOBEC3G is incorporated into HIV-1 through its interaction with Gag NC, and it is therefore possible that APOBEC3G might inhibit tRNA^{Lys3} annealing through its binding to NC.

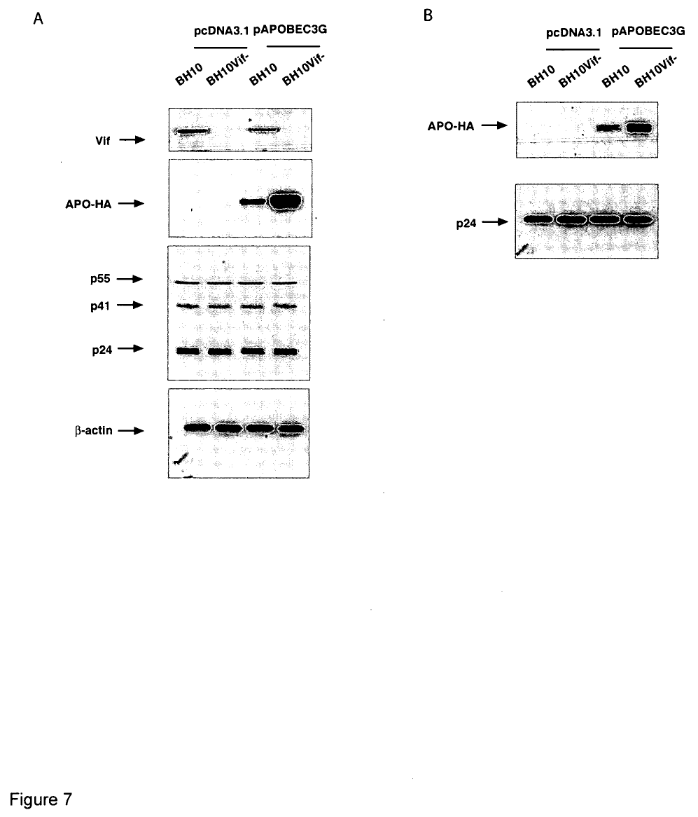

Briefly, the extracellular viruses were isolated, and protein composition of the different cell lysates and the virions produced from these cells is analyzed by the Western blots in FIG. 7, A and B, respectively. The panels, moving down from the top panel, are probed, respectively, with anti-Vif, anti-HA (which detects APOBEC3G tagged with HA), anti-capsid (CA), and anti-β-actin. Using aliquots of cell lysates containing equal amounts of β-actin (FIG. 7A, panel 4), these results show that cells expressing BH10Vif− viral proteins contain the normal pattern of viral Gag and capsid proteins (FIG. 7A, panel 3), but lack Vif (FIG. 7A, panel 1). Vif facilitates the proteosomal degradation of APOBEC3G (23), and as previously described, the absence of Vif in the cell results in a higher cellular concentration of APOBEC3G (FIG. 7A, panel 2). The results shown in FIG. 7B represent Western blots of lysates of viruses produced from these cells, and show that in the presence of cellular APOBEC3G, but in the absence of cellular Vif, the virions produced contain increased amounts of APOBEC3G.

293T cells were cotransfected with plasmid containing BH10 or BH10Vif− DNA and with either pcDNA3.1 alone or containing DNA coding for human hA3G. Thus four types of viruses are produced: wild-type viruses (BH10) in the absence or presence of hA3G, and Vif-negative viruses in the absence or presence of hA3G. The protein composition of lysates of the different cells and extracellular virions produced from them is shown in the Western blots in FIG. 7. Cells expressing BH10Vif− viral proteins contain the normal pattern of viral Gag and capsid proteins found in BH10, but for virions lacking Vif, the cellular expression and viral incorporation of hA3G is much higher^{22,23}. There is also no change in the ability of tRNA^{Lys3} to be selectively packaged into all four types of virions. Total viral RNA was extracted from the virions, and analyzed by dot-blot hybridization with probes specific for tRNA^{Lys3} or viral genomic RNA, as previously described (56). The data in Table 1 show no difference-in the tRNA^{Lys3}:genomic RNA ratios found for the four viral types.

TABLE 1
tRNA^{Lys3} and genomic RNA incorporation into HIV-1.

| | pcDNA3.1 | | pAPOBEC3G | |
|---|---|---|---|---|
| | BH10 | BH10Vif- | BH10 | BH10Vif- |
| Genomic RNA | 1.00 | 0.97 | 0.99 | 0.98 |
| tRNA^{Lys3} | 1.00 | 1.01 | 0.98 | 0.97 |

To study in vivo tRNA^{Lys3} annealing to viral RNA and the ability of the annealed tRNA^{Lys3} to initiate reverse transcription, total viral RNA was isolated and used as the source of the primer tRNA^{Lys3} annealed to viral genomic RNA in vivo, in an in vitro reverse transcription assay. The assumption that the annealed primer tRNA in the total viral RNA reflects its annealed configuration in vivo rests upon several pieces of evidence. Earlier studies have reported that the annealed primer tRNA in retroviruses is thermally stable (61), and the inventors have similarly found that in the reverse transcription reaction buffer, the primer tRNA^{Lys3} bound to the viral RNA template is very heat-stable, dissociating only at temperatures above 70° C. (unpublished data). Second, unannealed tRNA^{Lys3} added to viral RNA under reverse transcription reaction conditions at 37° C. will not anneal to the genomic RNA (65, 63). Third, the amount of tRNA^{Lys3} annealed to viral RNA, in wild-type viruses, as measured by this method, is proportional to the amount of tRNA^{Lys3} packaged into the virion (60). Fourth, the different degrees of inhibition of tRNA^{Lys3} annealing produced in virions containing wild type or mutant Gag (62) must reflect what had occurred in the virus since the total viral RNA used in the in vitro reverse transcription reaction has been deproteinized. Fifth, although the total viral RNA used has been deproteinized, it has been shown that only a transient exposure of NC to total viral RNA is required to produce long-term effects upon tRNA^{Lys3} annealing to viral RNA (56). Sixth, a mutant tRNA^{Lys3} with an altered anticodon sequence (SUU to CUA) is an efficient primer for reverse transcription in vitro when it is heat-annealed to genomic RNA. However, while this mutant tRNA is packaged into HIV-1 in vivo, it does not act as a primer tRNA in our RT assay using total viral RNA unless we first heat-denature the total viral RNA and allow the tRNA to anneal back to the genomic RNA (63).

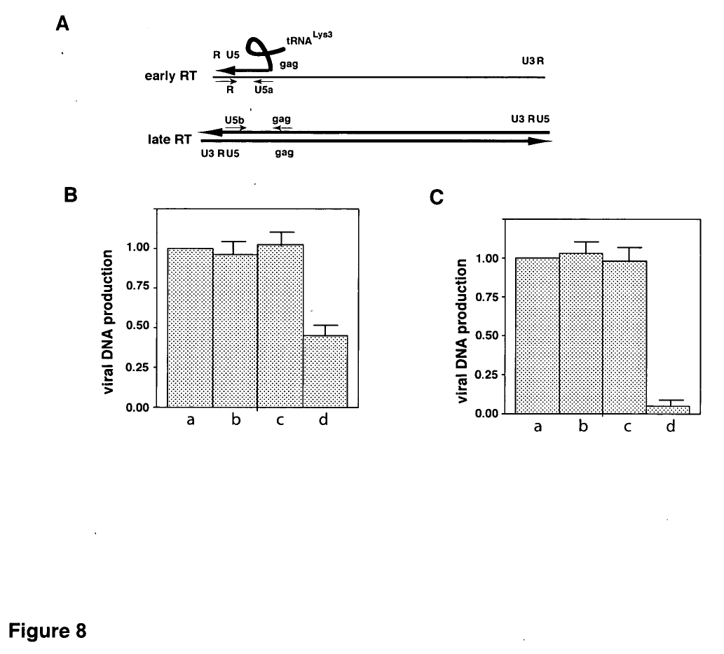

The viral DNA content in the permissive T lymphocyte cell line SupT1 infected with equal amounts of one of the 4 types of virions was next examined. Both early minus strand strong stop (−SS) DNA (R-U5) synthesis and late (U5-gag) DNA synthesis were monitored over the 24 hours post-infection using real-time fluorescence-monitored PCR, and the results are graphed in FIG. 8. The RT/PCR-amplified regions of viral DNA examined are shown in panel A of FIG. 8. As previously reported^{16,67,68}, it is shown that in cells infected with Vif-negative HIV-1 exposed to hA3G, the production of −SS DNA synthesis is reduced to about 45% that of wild-type viruses, while the production of late viral DNA sequences is reduced to 5% of that produced in wild-type viruses.

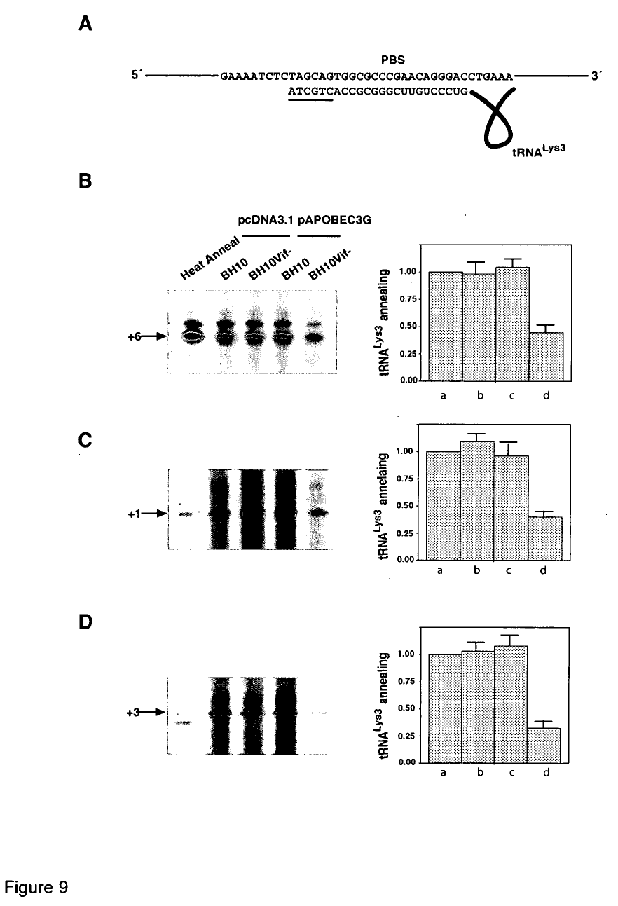

tRNA^{Lys3} annealing to viral RNA was measured using total viral RNA in an in vitro reverse transcription assay as the source of the primer tRNA^{Lys3} annealed to viral genomic RNA in vivo^{56,64}. FIG. 9A shows the 3′ terminal 18 nucleotides of tRNA^{Lys3} annealed to a complementary region near the 5′ terminus of viral RNA known as the primer binding site (PBS). Also shown are the first 6 deoxynucleotides added to the 3′ terminus of tRNA^{Lys3} during the initiation of reverse transcription, in the order 5′CTGCTA3′. FIG. 9B shows the radioactive tRNA^{Lys3} extended by 6 bases in the presence of ddATP, resolved by 1D PAGE. There is also a slower moving tRNA extension product which may represent misincorporation at position 6 rather than ddATP, which will result in ddATP being incorporated at a later position in the DNA. Lane 1 represents purified human placental tRNA^{Lys3} heat-annealed in vitro to synthetic viral genomic RNA. Lanes 2 through 5 use total viral RNA isolated from the 4 types of virions as the source of primer/template. These results, shown graphically in the right side of the panel, indicate that tRNA^{Lys3} annealing is reduced approximately 55% when Vif-negative virions are produced from 293T cells expressing hA3G (lane 5).

The tRNA^{Lys3} annealed to the viral RNA in vivo is found in two states in the viruses: unextended, and two base extended⁵⁶. These can be separately detected by measuring the ability of the total viral RNA to incorporate either dCTP (FIG. 9C) or dGTP (FIG. 9D). Resolution of the one and three base extension products by 1D PAGE again indicates a reduction in the amount of annealed tRNA^{Lys3} present in Vif-negative virions produced from 293T cells expressing hA3G, and these reductions are presented graphically in the right side of panels C and D. The data indicate a significant reduction (55-70%) in the amount of annealed tRNA^{Lys3} present in Vif-negative virions produced from 293T cells expressing hA3G.

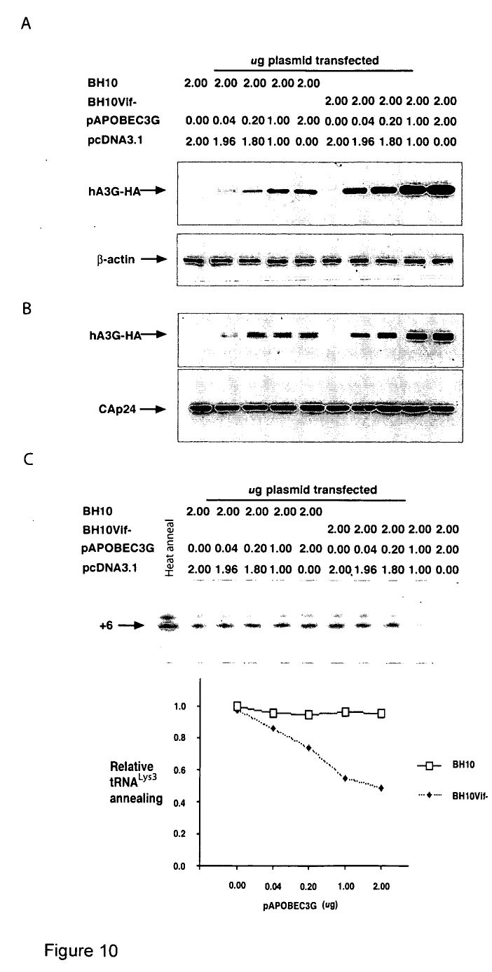

As shown in FIG. 10, the inhibition of tRNA^{Lys3} annealing in BH10Vif-negative viruses produced in non-permissive 293T cells is dependent upon the amount of hA3G expressed in the cell and incorporated into the virus. Both wild-type and Vif-negative viruses were produced in the absence or presence of increasing amounts of hA3G. Western blot analysis of cell (A) or viral (B) lysates show that while 293T cells cotransfected with both HIV-1 DNA and increasing amounts of pAPOBEC3G show an increase in hA3G in the cell, this increase is much larger when the viruses are not able to express Vif (3A). FIG. 10B shows that the amount of hA3G incorporated into the virus is proportional to the amount expressed in the cell.

Total viral RNA was isolated from these different virions, and the amount of annealed tRNA^{Lys3} was measured as described for the experiment shown in FIG. 9B. The upper part of FIG. 10C shows the 6 base-extended products resolved by 1D-PAGE. The electrophoretic bands were quantitated by phosphorimaging (BioRad), and the results, plotted in the bottom part of FIG. 10C, show an inverse correlation between the ability of hA3G to get into the virion and the amount of tRNA^{Lys3} annealed.

It has been previously reported reported that neither HIV-1 RNA^{16,17} nor tRNA^{Lys3 71} undergo hA3G-induced deamination. This conclusion was verified through sequencing of both RT/PCR products of gel-purified viral tRNA^{Lys3 28} and RT/PCR products representing viral RNA sequences starting at the C₁₅ in the R region and ending immediately after stem loop 3 of the leader sequence, which represent any known sequences in viral RNA postulated to be involved in tRNA^{Lys3} annealing⁷². An investigation was carried out with either of the zinc coordination motifs in hA3G inactivated by mutations and revealed that while only the C-terminal site is actively involved in DNA deamination, the N-terminal site retains anti-viral function¹⁹. To further test the conclusion that deamination is not required for at least some of the anti-viral effects of hA3G, 293T cells were cotransfected with BH10Vif− DNA and DNA coding for an N-terminal fragment (hA3G1-156,containing amino acids 1-156) or a C-terminal fragment of hA3G (hA3G105-384, containing amino acids 104-384; see SEQ ID NO: 21 and FIG. 4 for a schematic representation of hA3G). Although both hA3G1-156 and hA3G105-384 each contain one zinc coordination motif, none is capable of G-A deamination mutations in viral DNA sequences 492-764, which contain sequences starting in the C₁₅ in the R region and ending immediately after stem loop 3 in the leader region of HIV-1 genome. The inability to deaminate this DNA is shown in Table 2. DNA was extracted from these cells, and PCR products representing DNA sequences 492-764 were sequenced and examined for mutations. While viral packaging of wild-type hA3G produces a total of 31 G-A mutations, in 6 clones sequenced, no G-A mutation is seen when virions package either hA3G1-156 or hA3G105-384.

TABLE 2
Viral DNA hypermutation and antiviral activity of wild-type and mutant APOBEC3G

| | | | | | | G → A | |
|---|---|---|---|---|---|---|---|
| | Total | Total | | | | Mutations | |
| | clones | Bases | Total | G → A | Other | per | Viral |
| APOBEC3G | sequenced | sequenced | Mutations | Mutations | Mutations | 100 bps | Infectivity |
| Control | 6 | 1632 | 2 | 0 | 2 | 0 | 100 |
| hA3G | 6 | 1632 | 32 | 31 | 1 | 2 | 9 |
| hA3G105-384 | 6 | 1632 | 1 | 0 | 1 | 0 | 32 |
| hA3G1-156 | 6 | 1632 | 2 | 0 | 2 | 0 | 38 |

The relative infectivity of the different viral types was measured by the MAGI assay⁷³. As shown in Table 2, wild-type hA3G reduces infectivity of BH10Vif− virions >90%, while the Nand C-terminal fragments in the virions reduce viral infectivity by >60% and 70%, respectively, as compared to that achieved by BH10Vif− in the absence of hA3G.

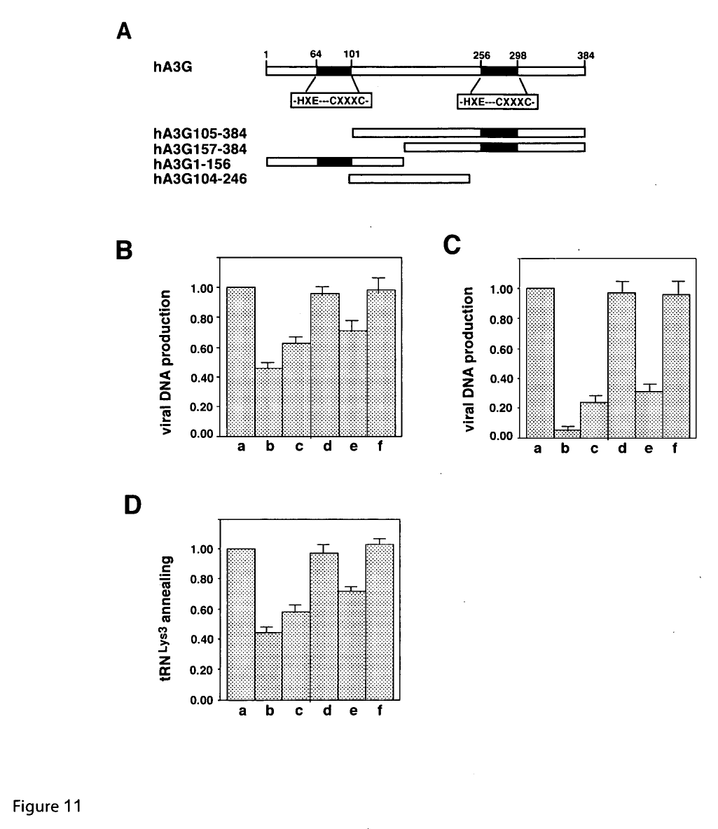

The ability of mutant forms of hA3G to inhibit early and late DNA synthesis, and tRNA^{Lys3} annealing was examined next. The mutant forms of hA3G used are shown in FIG. 11, panel A. These mutant species were previously used to map the site on hA3G required for its viral incorporation to (amino acids 104-156; SEQ ID NO: 1, as described above). The cellular expression and viral incorporation of these truncated species was also reported above, except for hA3G104-246, which is incorporated efficiently into virions (data not shown). Using real-time fluorescence-monitored PCR, as described for FIG. 8, the effect of the expression of mutant forms of hA3G on both early minus strand strong stop (−SS) DNA synthesis (panel B), and late viral DNA synthesis (panel C) was monitored over 24 hours post-infection. The results are shown graphically in FIG. 11B,C. Both hA3G1-156 and hA3G105-384 reduce early and late DNA synthesis, although not as strongly as the reductions due to full-length hA3G. hA3G105-384 has somewhat stronger inhibitory powers than hA3G1-156. If amino acids 104-156 are missing from the C-terminal fragment (hA3G157-384), no inhibition of viral DNA synthesis is seen, since this fragment is not incorporated into the virion, as described above. Also, hA3G missing both N- and C-terminal sequences containing the zinc coordination motifs (hA3G104-246) is not able to inhibit viral DNA synthesis, although it is incorporated into the virions (data not shown).

To measure tRNA^{Lys3} annealing, total viral RNA was isolated from these different virions, and the amount of extendable annealed tRNA^{Lys3} was measured as described for the experiment shown in FIG. 9B. The electrophoretic bands were quantitated by phosphorimaging (BioRad), and the results plotted in FIG. 11D, were normalized to that found for BH10Vif− lacking hA3G sequences. Both hA3G1-156 and hA3G105-384 inhibit tRNA^{Lys3} annealing, although less so than full-length hA3G. The C-terminal fragment inhibits annealing slightly more than the N-terminal fragment. Mutant hA3G, unable to be incorporated into virions (hA3G157-384), shows no ability to inhibit tRNA^{Lys3} annealing similarly to hA3G104-246, which lacks both N- and C-terminal regions. A strong correlation between the ability of wild-type and mutant hA3G to inhibit tRNA^{Lys3} annealing and their ability to inhibit early and late viral DNA synthesis can be observed by comparing panels B, C, and D. While inhibition of tRNA^{Lys3} annealing seems to be a likely cause of reduction in early DNA synthesis, the cause of reduction in late DNA production remains to be determined.

### EXAMPLE 8

### Rescue of APOBEC3G-Induced Inhibition of tRNA^{Lys3}-Primed Initiation of Reverse Transcription of Nucleocapsid

The total viral RNA was pre-incubated with 10 pmolar recombinant HIV-1 nucleocapsid protein (NCp7) in reverse transcription buffer at 37° C. for 30 min. The NCp7 was then removed by proteinase K digestion and phenol-chloroform extraction. The RNA was then used as the source of primer/template in the reverse transcription reaction, and the tRNA^{Lys3} extension products were analyzed by 1D PAGE. The results indicate that the reduced initiation of reverse transcription seen in Vif-negative viruses produced from 293T cells expressing APOBEC3G is rescued 40-70% when the total viral RNA is transiently exposed to mature nucleocapsid protein. Exposure to nucleocapsid of the total viral RNA isolated from wild-type viruses produced in APOBEC3G-expressing cells has no effect upon initiation of reverse transcription.

### EXAMPLE 9

### Cellular Expression of Human APOBEC3G-Derived Peptides Inhibits HIV-1 Replication by Preventing Vif-Medicated APOBEC3G Degradation

Recent studies demonstrate that non-permissive cells, such as H9 cells, contain a protein called hA3G which prevents HIV-1 replication in the absence of Vif (13). hA3G belongs to an APOBEC superfamily containing at least 10 members, which share a cytidine deaminase motif (a conserved His-X-Glu and Cys-X-X-Cys Zn²⁺ coordination motif) (14). Vif is able to bind to hA3G (20), and can reduce both the cellular expression of hA3G and its incorporation into virions (21). The reduction in cellular expression has been attributed to both inhibition of hA3G translation and its degradation in the cytoplasm by Vif (22). Several lines of evidence have established that Vif induces the rapid degradation of hA3G by a proteasome-dependent mechanism, and the proteasome inhibitors prevent the Vif-mediate down-modulation of hA3G, resulting in restoring the virion encapsidation of hA3G.

The inhibition of Vif-mediated hA3G degradation suggests a new anti-HIV-1 target for drug development, and the mechanism of this inhibition is herein investigated to that effect. A removal of the N-terminal 104 amino acids or the C-terminal 245-384 amino acid residues of hA3G was carried out and shown to have no effect on Vif-mediated degradation, whereas the deletion of the N-terminal 156 amino acids abolished the sensitivity to Vif action and ability to bind to Vif. The C-terminal linker sequence in hA3G, amino acids 157-245, is also required for the Vif mediated degradation, but not for interaction between hA3G and Vif. Expression of hA3G-derived peptides neutralize these Vif function, and inhibit the HIV-1 replication in a non-permissive cell line. The data presented herein suggest that the binding of Vif to hA3G is required, but not sufficient for hA3G degradation. The C-terminal linker sequence plays an important role in the Vif-mediated degradation, possibly through interaction with other cofactors required for the process. As a novel anti-HIV strategy, hA3G-derived peptides can be used to block the Vif's function, resulting in the inhibition of HIV-1 replication.

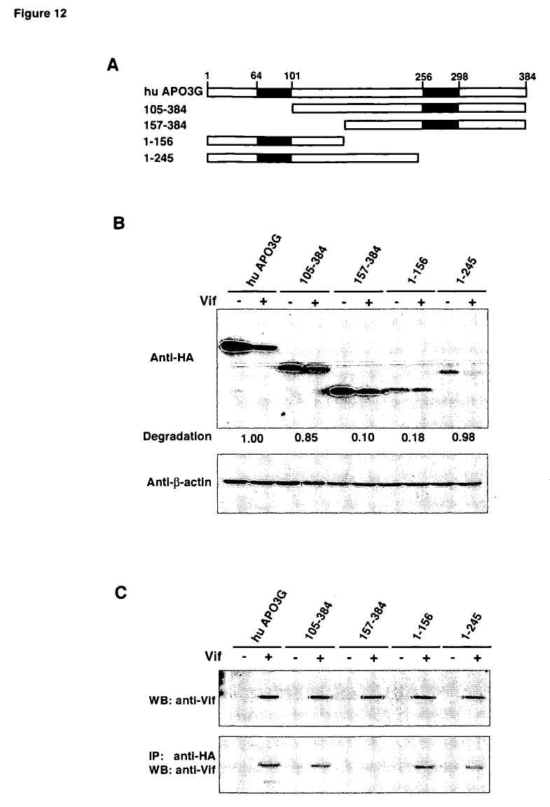

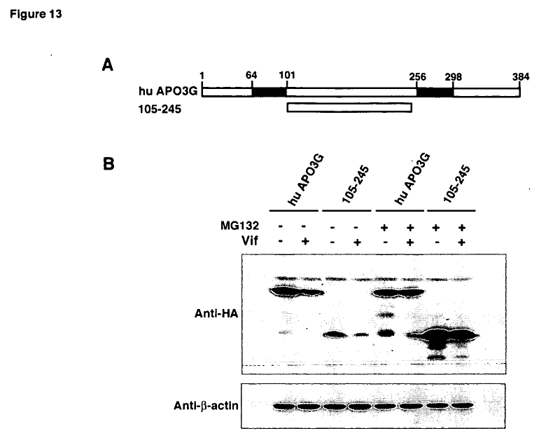

Although the fact that Vif interacts with cytoplasmic hA3G as part of a Vif-Cul5-SCF complex, resulting in the ubiquination of hA3G and its degradation is known (23), the motifs within human hA3G which are involved in the depletion are still unclear. To address the question, a series of hA3G truncations were constructed, as graphically represented in FIG. 12A, and used to transfect 293T cell, or co-transfect with a plasmid coding for HIV-1 Vif. The cytoplasmic expression of the different hA3G variants in the presence or absence of Vif was determined by Western blots probed with anti-HA and anti-β-actin. As shown in FIG. 12B, full-length hA3G and N-terminal truncations were well expressed, while C-terminal truncations of hA3G appeared reduced in expression, even in the absence of Vif (upper panel), consistent with the results presented for example in Example 7. Vif alone is sufficient for triggering the degradation of human hA3G (FIG. 12B, lane 2). The deletion of the N-terminal 104 amino acids or the C-terminal 246-384 amino acids does not significantly affect their ability to be degraded by Vif, whereas deletions of the N-terminal 156 amino acids or C-terminal 157-384 amino acids appear to make hA3G resistant to Vif-mediated degradation. To further analyze the effect of N- or C-terminal deletions of hA3G upon Vif-mediated degradation, the ratio of expression of the different hA3G variants in the presence or absence of Vif was determined, and normalized to a ratio of 1.00 for wild-type hA3G. These ratios are listed at the bottom of upper panel (FIG. 13B). The study shows that the deletion of the N-terminal 156 or C-terminal 157-384 amino acids results in 80% and 90% reduction in the ability of the resulting fragments to be degraded by Vif, respectively, while only minor decreases were found among other truncated forms of hA3G. These results indicate that the amino acid sequence 105-245 of SEQ ID NO: 21, comprising the linker sequence between the two zinc coordination motifs in hA3G, is required for Vif-mediated degradation.

The ability of the different hA3G variants to bind to Vif was assessed by co-immunoprecipitation. As shown in FIG. 12C, only the deletion of the N-terminal 156 amino acids of hA3G abolishes the association with Vif, confirming the results shown above that the N-terminal linker sequence, i.e., amino acids 105-156, is involved in the association between hA3G and Vif. The results of FIG. 12 also suggest that the resistance to Vif-mediated degradation of the C-terminal fragment, hA3G 157-384, might be a result of its failure to bind to Vif. Furthermore, these data also indicate that the association with Vif is not sufficient for the degradation of human hA3G, i.e., the N-terminal fragment, hA3G 1-156 is able to bind to Vif, but its expression is not affected by the presence of Vif.

Thus, as shown here and above the linker sequence between the two zinc coordination motifs in hA3G is involved in Vif-mediated degradation. To further explore the role of this fragment in this degradation, the cytoplasmic expression of the linker fragment hA3G 105-245 in the presence or absence of Vif was examined. The results (FIG. 13, left panel) show that the expression of this fragment is reduced by Vif, to a level similar as the reduction of wild type hA3G, suggesting that the linker sequence between two zinc coordination motifs is sufficient for the Vif-mediated degradation. The addition of a proteasome inhibitor, MG132, restored the expression of both wild type and hA3G104-245 in the presence of Vif (FIG. 13, right panel), thereby confirming that the decrease in expression of the linker fragment resulted from the proteasomal-dependent degradation induced by Vif.

The presence of Vif can reduce the expression of hA3G105-245 (FIG. 13), but not hA3G 1-156 (FIG. 12), which is able to bind to Vif. These results suggest that the C-terminal linker sequence between the two zinc coordination motifs, hA3G 157-245, is also involved in the Vif-mediated degradation, although this fragment is not required for the interaction between Vif and hA3G.

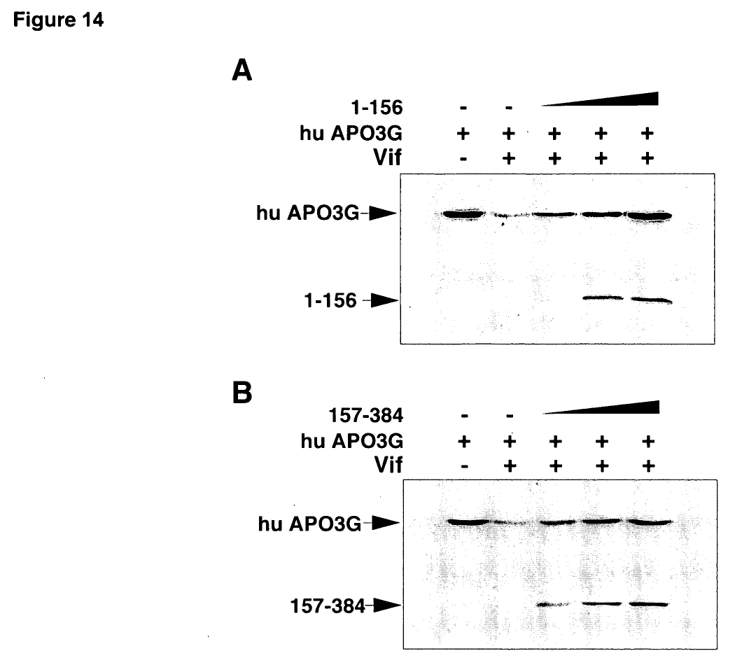

The effect of different hA3G fragments upon Vif-mediated degradation of full-length hA3G was next examined. 293T cells were co-transfected with plasmids coding for Vif and full-length human hA3G, and increasing amounts of plasmids expressing hA3G1-156 or hA3G157-384. As shown in FIG. 14, an increase in the cytoplasmic expression of full-length hA3G was detected with an increase in expression of hA3G 1-156 or hA3G 157-384, respectively. However, a co-transfection of the same amount of control plasmid pcDNA3.1, had no effect on the expression of full-length hA3G (data not shown). These results indicate that both the N-terminal and C-terminal fragments can dominantly block the Vif-mediated degradation of full-length hA3G.

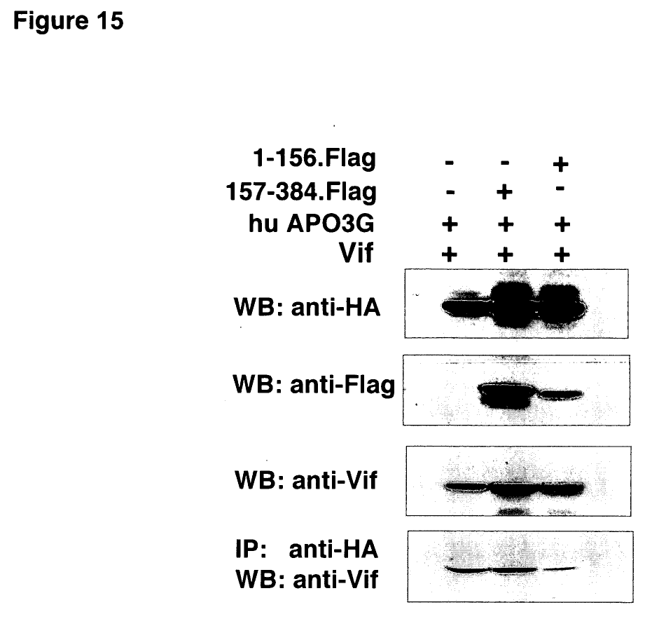

Next, 293T cell were co-transfected with plasmids coding for Vif, HA-tagged full-length human hA3G, and Flag-tagged hA3G 1-156 or hA3G 157-384. The effect of these hA3G fragments on the association of Vif and full-length wild-type hA3G was analyzed, using co-immunoprecipition with anti-HA to coimmunoprecipitate the complexes. The results indicated that a reduced amount of Vif was pulled down with full-length hA3G, using anti-HA, when Flag-tagged hA3G 1-156, but not when hA3G 157-384 was expressed (FIG. 15), suggesting that the blocking effect of hA3G 1-156 on the degradation results from a competitive binding with full-length hA3G to Vif. Interestingly, hA3G 157-384 can dominantly inhibit the Vif-mediated degradation (FIG. 15A), even though it is unable to bind to Vif (FIG. 12C), or to interrupt the interaction between Vif and hA3G (FIG. 15B). It was hypothesized that hA3G 157-245 might interact with some unknown cellular factors required for Vif-mediated degradation, and that overexpressing hA3G 157-245 might compete with full-length hA3G to bind to these factors, thereby inhibiting the degradation.

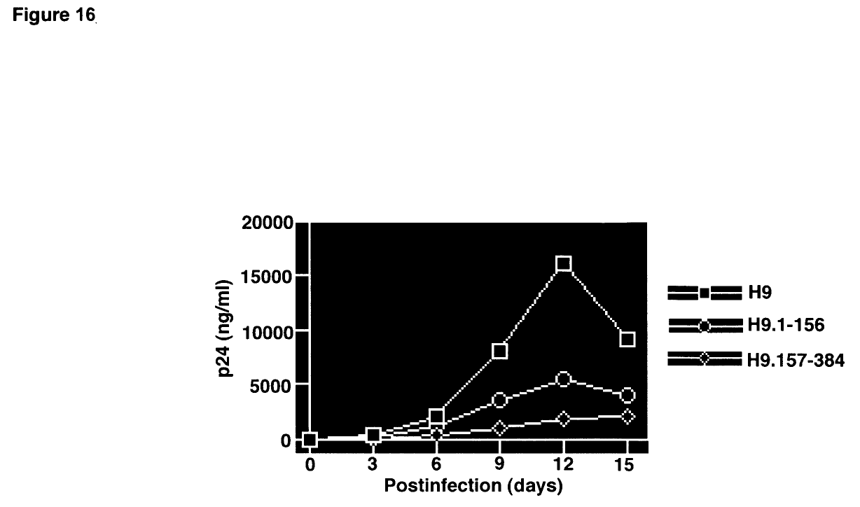

Transient expression of hA3G fragments that block Vif-mediated degradation might also inhibit HIV-1 replication. To investigate this, stable H9 cell lines were established that constitutively expressed either hA3G 1-156 or hA3G 157-384. The cytoplasmic expression of the two fragments in H9 was determined by Western blots probed with anti-HA. These cell lines were then infected with wild-type BH10 HIV-1, and extracellular p24 was measured as a sign of viral production. As shown in FIG. 16, the amount of extracellular p24 produced from BH10-infected H9 cells reached a maximum concentration at 12 days, while the production of p24 in the medium of infected H9 expressing either hA3G 1-156 or hA3G 157-384, was reduced to 34% and 12% of the control group, respectively, showing that either hA3G 1-156 or hA3G 157-384 inhibit HIV-1 replication in the non-permissive cell line H9 in the presence of Vif. Taken together, it has been demonstrated that hA3G-derived peptides can be used to neutralize Vif's function, resulting in the inhibition of HIV-1 replication.

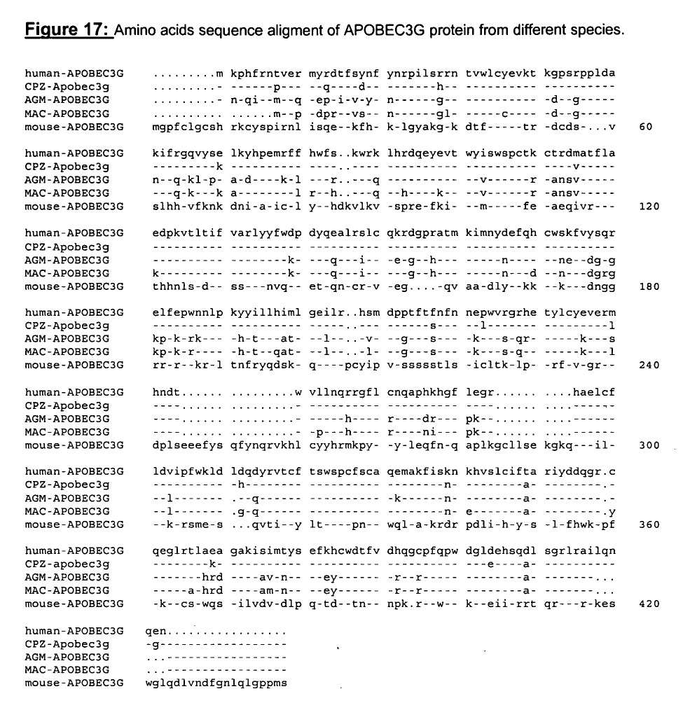

A recent work demonstrated that hA3G can also inhibit hepatitis B virus replication, independently of the molecules's cytidine deaminase activity (70). The mechanism of this activity is still unclear, but an attractive application of this finding is to use the hA3G-derived peptides according to the teachings of the present invention in anti-haepatitis B therapy. In any event, in view of the conservation of hA3G amongst species (FIG. 17) of the conservation of Gag amongst species and notable retroviruses (HBV is not a retrovirus), the present invention shows that peptides from hA3G and derivatives thereof are antiviral agents which can be used against HIV and other retroviruses and viruses.

Although the present invention has been described hereinabove by way of preferred embodiments thereof, it can be modified, without departing from the spirit and nature of the subject invention as defined in the appended claims.

What is claimed is:

1. A method of treating or preventing viral infections by inhibiting tRNA^{Lys3} annealing and/or priming on a viral genome thereby reducing viral replication.

2. A purified polypeptide comprising amino acids 104-156 of APOBEC3G having the ability, when introduced in a viral particle, to inhibit tRNA^{Lys3} annealing and/or priming on a viral genome, thereby reducing viral replication.


SEQUENCE LISTING (47 sequences)

SEQ ID NO: 1 - protein, 53 aa, Homo sapiens
MATFLAEDPKVTLTIFVARLYYFWDPDYQEALRSLCQKRDGPRATMKIMNYDE

SEQ ID NO: 2 - DNA, 33 bp, Artificial
  Synthetic construct
agatcattagggatttaggaaaacagatggcag

SEQ ID NO: 3 - DNA, 33 bp, Artificial
  Synthetic construct
ctgccatctgttttcctaaatccctaatgatct

SEQ ID NO: 4 - DNA, 31 bp, Artificial
  Synthetic construct
gccagaattcaaggatgaagcctcacttcag

SEQ ID NO: 5 - DNA, 65 bp, Artificial
  Synthetic construct
tagaagctcgagtcaagcgtaatctggaacatcgtatggatagttttcctgattctggagaatgg

SEQ ID NO: 6 - DNA, 30 bp, Artificial
  Synthetic construct
taagcggaattcatgaagcctcacttcaga

SEQ ID NO: 7 - DNA, 30 bp, Artificial
  Synthetic construct
tagaagctcgagtcaagcgtaatctggaac

SEQ ID NO: 8 - DNA, 33 bp, Artificial
  Synthetic construct
taggcggaattcatggtgtattccgaacttaag

SEQ ID NO: 9 - DNA, 33 bp, Artificial
  Synthetic construct
taagtcgaattcatggccacgttcctggccgag

SEQ ID NO: 10 - DNA, 32 bp, Artificial
  Synthetic construct
taagtcgaattcatgtttcagcactgtggagc

SEQ ID NO: 11 - DNA, 60 bp, Artificial
  Synthetic construct
tagaagctcgagtcaagcgtaatctggaacatcgtatggatattcgtcataattcatgat

SEQ ID NO: 12 - DNA, 60 bp, Artificial
  Synthetic construct
tagaagctcgagtcaagcgtaatctggaacatcgtatggatactggttgcatagaaagcc

SEQ ID NO: 13 - DNA, 60 bp, Artificial
  Synthetic construct
tagaagctcgagtcaagcgtaatctggaacatcgtatggatagatgcacaggctcacgtg

SEQ ID NO: 14 - DNA, 33 bp, Artificial
  Synthetic construct
ataatagtcgacatgggcgcccgcgccagcgtg

SEQ ID NO: 15 - DNA, 30 bp, Artificial
  Synthetic construct
gactggtctagaagggcctccttcagctgg

SEQ ID NO: 16 - DNA, 33 bp, Artificial
  Synthetic construct
gcggcggtcgacatgcccatcgtgcagaacatc

SEQ ID NO: 17 - DNA, 33 bp, Artificial
  Synthetic construct
gcggcgtctagattacaggatgctggtggggct

SEQ ID NO: 18 - DNA, 33 bp, Artificial
  Synthetic construct
gcggcgtctagattacatgatggtggcgctgtt

SEQ ID NO: 19 - DNA, 33 bp, Artificial
  Synthetic construct
gcggcgtctagattaaaaattagcctgtcgctc

SEQ ID NO: 20 - DNA, 1717 bp, Homo sapiens
ctgccagggggagggccccagagaaaaccagaaagagggtgagagactgaggaagataaagcgtcccagggcctcctacaccagcgcctgagcaggaagcgggaggggccatgactacgaggccctgggaggtcactttagggagggctgtcctaaaaccagaagcttggagcagaaagtgaaaccctggtgctccagacaaagatcttagtcgggactagccggccaaggatgaagcctcacttcagaaacacagtggagcgaatgtatcgagacacattctcctacaacttttataatagacccatcctttctcgtcggaataccgtctggctgtgctacgaagtgaaaacaaagggtccctcaaggccccctttggacgcaaagatctttcgaggccaggtgtattccgaacttaagtaccacccagagatgagattcttccactggttcagcaagtggaggaagctgcatcgtgaccaggagtatgaggtcacctggtacatatcctggagcccctgcacaaagtgtacaagggatatggccacgttcctggccgaggacccgaaggttaccctgaccatcttcgttgcccgcctctactacttctgggacccagattaccaggaggcgcttcgcagcctgtgtcagaaaagagacggtccgcgtgccaccatgaagatcatgaattatgacgaatttcagcactgttggagcaagttcgtgtacagccaaagagagctatttgagccttggaataatctgcctaaatattatatattactgcacatcatgctgggggagattctcagacactcgatggatccacccacattcactttcaactttaacaatgaaccttgggtcagaggacggcatgagacttacctgtgttatgaggtggagcgcatgcacaatgacacctgggtcctgctgaaccagcgcaggggctttctatgcaaccaggctccacataaacacggtttccttgaaggccgccatgcagagctgtgcttcctggacgtgattcccttttggaagctggacctggaccaggactacagggttacctgcttcacctcctggagcccctgcttcagctgtgcccaggaaatggctaaattcatttcaaaaaacaaacacgtgagcctgtgcatcttcactgcccgcatctatgatgatcaaggaagatgtcaggaggggctgcgcaccctggccgaggctggggccaaaatttcaataatgacatacagtgaatttaagcactgctgggacacctttgtggaccaccagggatgtcccttccagccctgggatggactagatgagcacagccaagacctgagtgggaggctgcgggccattctccagaatcaggaaaactgaaggatgggcctcagtctctaaggaaggcagagacctgggttgagcctcagaataaaagatcttcttccaagaaatgcaaacaggctgttcaccaccatctccagctgatcacagacaccagcaaagcaatgcactcctgaccaagtagattcttttaaaaattagagtgcattactttgaatcaaaaatttatttatatttcaagaataaagtactaagattgtgctcaatacacagaaaagtttcaaacctactaatccagcgacaatttgaatcggttttgtaggtagaggaataaaatgaaatactaaatctttctgtaaaaaaaaaa

SEQ ID NO: 21 - protein, 384 aa, Homo sapiens
MKPHFRNTVERMYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQNQEN

SEQ ID NO: 22 - DNA, 1521 bp, Homo sapiens
atgggcgcccgcgccagcgtgctgagcggcggcgagctggaccgctgggagaagatccgcctgcgccccggcggcaagaagaagtacaagctgaagcacatcgtgtgggccagccgcgagctggagcgcttcgccgtgaaccccggcctgctggagaccagcgagggctgccgccagatcctgggccagctgcagcccagcctgcagaccggcagcgaggagctgcgcagcctgtacaacaccgtggccaccctgtactgcgtgcaccagcgcatcgagatcaaggacaccaaggaggccctggacaagatcgaggaggagcagaacaagagcaagaagaaggcccagcaggccgccgccgacaccggccacagcaaccaggtgagccagaactaccccatcgtgcagaacatccagggccagatggtgcaccaggccatcagcccccgcaccctgaacgcctgggtgaaggtggtggaggagaaggccttcagccccgaggtgatccccatgttcagcgccctgagcgagggcgccaccccccaggacctgaacaccatgctgaacaccgtgggcggccaccaggccgccatgcagatgctgaaggagaccatcaacgaggaggccgccgagtgggaccgcgtgcaccccgtgcacgccggccccatcgcccccggccagatgcgcgagccccgcggcagcgacatcgccggcaccaccagcaccctgcaggagcagatcggctggatgaccaacaacccccccatccccgtgggcgagatctacaagcgctggatcatcctgggcctgaacaagatcgtgcgcatgtacagccccaccagcatcctggacatccgccagggccccaaggagcccttccgcgactacgtggaccgcttctacaagaccctgcgcgccgagcaggccagccaggaggtgaagaactggatgaccgagaccctgctggtgcagaacgccaaccccgactgcaagaccatcctgaaggccctgggccccgccgccaccctggaggagatgatgaccgcctgccagggcgtgggcggccccggccacaaggcccgcgtgctggccgaggccatgagccaggtgaccaacagcgccaccatcatgatgcagcgcggcaacttccgcaaccagcgcaagatcgtgaagtgcttcaactgcggcaaggagggccacaccgcccgcaactgccgcgccccccgcaagaagggctgctggaagtgcggcaaggagggccaccagatgaaggactgcaccgagcgacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagatagggggccagctga

SEQ ID NO: 23 - protein, 501 aa, Homo sapiens
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMMQRGNFRNQRKIVKCFNCGKEGHTARNCRAPRKKGCWKCGKEGHQMKDCTERQANFLGKIWPSHKGRPGNFLQSRPEPTAPPEESFRFGEETTTPSQKQEPIDKELYPLASLRSLFGSDPSSQZ

SEQ ID NO: 24 - protein, 9 aa, Artificial
  Synthetic construct
HXEXPPXXC

SEQ ID NO: 25 - DNA, 24 bp, Artificial
  Synthetic construct
ttagaccagatctgagcctgggag

SEQ ID NO: 26 - DNA, 24 bp, Artificial
  Synthetic construct
gggtctgagggatctctagttacc

SEQ ID NO: 27 - DNA, 22 bp, Artificial
  Synthetic construct
tgtgtgcccgtctgttgtgtga

SEQ ID NO: 28 - DNA, 21 bp, Artificial
  Synthetic construct
gagtcctgcgtcgagagagct

SEQ ID NO: 29 - DNA, 22 bp, Artificial
  Synthetic construct
ccagatctgagcctgggagctc

SEQ ID NO: 30 - DNA, 21 bp, Artificial
  Synthetic construct
ctccttctagcctccgctatc

SEQ ID NO: 31 - DNA, 43 bp, Artificial
  Synthetic construct
ttgaattcgcattgagcacctgcttttttttttttttttttgg

SEQ ID NO: 32 - DNA, 23 bp, Artificial
  Synthetic construct
tctttagtgagggttaattgccn

SEQ ID NO: 33 - DNA, 23 bp, Artificial
  Synthetic construct
ttgaattcgcattgagcacctgc

SEQ ID NO: 34 - DNA, 21 bp, Artificial
  Synthetic construct
ggcaattaaccctcactaaag

SEQ ID NO: 35 - DNA, 22 bp, Artificial
  Synthetic construct
ccagatctgagcctgggagctc

SEQ ID NO: 36 - DNA, 22 bp, Artificial
  Synthetic construct
ctccttctagcctccgctagtc

SEQ ID NO: 37 - DNA, 39 bp, Artificial
  Synthetic construct
gaaaatctctagcagtggcgcccgaacagggacctgaaa

SEQ ID NO: 38 - DNA, 24 bp, Artificial
  Synthetic construct
gucccuguucgggcgccactgcta

SEQ ID NO: 39 - protein, 384 aa, Homo sapiens; identical to SEQ ID NO: 21

SEQ ID NO: 40 - protein, 429 aa, Pan troglodytes
MGPFCLGCSHRKCYSPIRNLISQETFKFHFKNLGYAKGRKDTFLCYEVTRKDCDSPVSLHHGVFKNKDNIHAEICFLYWFHDKVLKVLSPREEFKITWYMSWSPCFECAEQIVRFLATHHNLSLDIFSSRLYNVQDPETQQNLCRLVQEGAQVAAMDLYEFKKCWKKFVDNGGRRFRPWKRLLTNFRYQDSKLQEILRPCYIPVPSSSSSTLSNICLTKGLPETRFCVEGRRMDPLSEEEFYSQFYNQRVKHLCYYHRMKPYLCYQLEQFNGQAPLKGCLLSEKGKQHAEILFLDKIRSMELSQVTITCYLTWSPCPNCAWQLAAFKRDRPDLILHIYTSRLYFHWKRPFQKGLCSLWQSGILVDVMDLPQFTDCWTNFVNPKRPFWPWKGLEIISRRTQRRLRRIKESWGLQDLVNDFGNLQLGPPMS

SEQ ID NO: 41 - protein, 384 aa, Cercopithecus aethiops
MKPHFRNPVERMYQDTFSDNFYNRPILSHRNTVWLCYEVKTKGPSRPPLDAKIFRGQVYSKLKYHPEMRFFHWFSKWRKLHRDQEYEVTWYISWSPCTKCTRDVATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTSNFNNELWVRGRHETYLCYEVERLHNDTWVLLNQRRGFLCNQAPHKHGFLEGRHAELCFLDVIPFWKLDLHQDYRVTCFTSWSPCFSCAQEMAKFISNNKHVSLCIFAARIYDDQGRCQEGLRTLAKAGAKISIMTYSEFKHCWDTFVDHQGCPFQPWDGLEEHSQALSGRLRAILQNQGN

SEQ ID NO: 42 - protein, 370 aa, Macaca mulatta
MVEPMDPRTFVSNFNNRPILSGLNTVWLCCEVKTKDPSGPPLDAKIFQGKVYSKAKYHPEMRFLRWFHKWRQLHHDQEYKVTWYVSWSPCTRCANSVATFLAKDPKVTLTIFVARLYYFWKPDYQQALRILCQKRGGPHATMKIMNYNEFQDCWNKFVDGRGKPFKPRNNLPKHYTLLQATLGELLRHLMDPGTFTSNFNNKPWVSGQHETYLCYKVERLHNDTWVPLNQHRGFLRNQAPNIHGFPKGRHAELCFLDLIPFWKLDGQQYRVTCFTSWSPCFSCAQEMAKFISNNEHVSLCIFAARIYDDQGRYQEGLRALHRDGAKIAMMNYSEFEYCWDTFVDRQGRPFQPWDGLDEHSQALSGRLRAI

SEQ ID NO: 43 - protein, 377 aa, Mus musculus
MNPQIRNMVEQMEPDIFVYYFNNRPILSGRNTVWLCYEVKTKDPSGPPLDANIFQGKLYPEAKDHPEMKFLHWFRKWRQLHRDQEYEVTWYVSWSPCTRCANSVATFLAEDPKVTLTIFVARLYYFWKPDYQQALRILCQERGGPHATMKIMNYNEFQHCWNEFVDGQGKPFKPRKNLPKHYTLLHATLGELLRHVMDPGTFTSNFNNKPWVSGQRETYLCYKVERSHNDTWVLLNQHRGFLRNQAPDRHGFPKGRHAELCFLDLIPFWKLDDQQYRVTCFTSWSPCFSCAQKMAKFISNNKHVSLCIFAARIYDDQGRCQEGLRTLHRDGAKIAVMNYSEFEYCWDTFVDRQGRPFQPWDGLDEHSQALSGRLRAI

SEQ ID NO: 44 - protein, 500 aa, Human immunodeficiency virus type 1
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMMQRGNFRNQRKIVKCFNCGKEGHTARNCRAPRKKGCWKCGKEGHQMKDCTERQANFLGKIWPSYKGRPGNFLQSRPEPTAPPEESFRSGVETTTPPQKQEPIDKELYPLTSLRSLFGNDPSSQ

SEQ ID NO: 45 - protein, 521 aa, Human immunodeficiency virus type 2
MGARSSVLRGKKVDELEKIRLRPGGKKKYRLKHIVWAANELGKFGLAESLLESKEGCQKIITVLDPLVPTGSENLKSLFNTVCVIWCLHAEEKVKDTEGAKQIVQRHLVAETGTADKMPSTSRPAAPPSGRGGNYPVQQIAGNYSHVPLSPRTLNAWVKLVEEKKFGAEVVPGFQALSEGCTPYDINQMLNCVGDHQAAMQIIREIINEEAADWDVQHPIPGPLPAGQLREPRGSDIAGTTSTVEEQIQWMFRAQNPIPVGNIYRRWIQIGLQKCVRMYNPTNILDVKQGPKEPFQSYVDRFYKSLRAEQTDPAVKNWMTQTLLVQNANPDCKLVLKGLGMNPTLEEMLTACQGIGGPGQKARLMAEALKEALAPAPIPFAAAQQRRTIKCWNCGKDGHSARQCRAPRRQGCWKCGKSGHVMANCPERQAGFLGIGPWGKKPRNFPVTRVPQGLTPTAPPADPAADLLEKYLQQGRKQKEQKMRPYKEVTEDLLHLEQGETPHKEATEDLLHLNSLFGKDQ

SEQ ID NO: 46 - protein, 513 aa, Simian immunodeficiency virus
MGGGHSALSGRSLDTFEKIRLRPNGKKKYQIKHLIWAGKEMERFGLHEKLLETKEGCQKIIEVLTPLEPTGSEGLKALFNLCCVIWCIHAEQKVKDTEEAVVTVKQHYHLVDKNEKAAKKKNETTAPPGGESRNYPVVNQNNAWVHQPLSPRTLNAWVKCVEEKRWGAEVVPMFQALSEGCLSYDVNQMLNVIGDHQGALQILKEVINEEAAEWDRTHRPPAGPLPAGQLRDPTGSDIAGTTSSIQEQIEWTFNANPRIDVGAQYRKWVILGLQKVVQMYNPQKVLDIRQGPKEPFQDYVDRFYKALRAEQAPQDVKNWMTQTLLIQNANPDCKLILKGLGMNPTLEEMLIACQGVGGPQHKAKLMVEMMSNGQNMVQVGPQKKGPRGPLKCFNCGKFGHMQRECKAPRQIKCFKCGKIGHMAKDCKNGQANFLGYGHWGGAKPRNFVQYRGDTVGLEPTAPPMETAYDPAKKLLQQYAEKGQRLREEREQTRKQKEKEVEDVSLSSLFGGDQ

SEQ ID NO: 47 - protein, 537 aa, Murine leukemia virus
MGQTVTTPLSLTLEHWGDVQRIASNQSVDVKKRRWVTFCSAEWPTFGVGWPQDGTFNLDIILQVKSKVFSPGPHGHPDQVPYIVTWEAIAYEPPPWVKPFVSPKLSPSPTGPILPSGPSTQPPPRSALYPALTPSIKPRPSKPQVLSDNGGPLIDLLTEDPPPYGEQGPSSSDGDGDREEATSTSEIPAPSPMVSRLRGKRDPPAADSTTSRVFPLRLGGNGQLQYWPFSSSDLYNWKNNNPSFSEDPGKLTALIESVLTTHQPTWDDCQQLLGTLLTGEEKQRVLLEARKAVRGNDGRPTQLPNEVNSAFPLERPDWDYTTPEGRNHLVLYRQLLLAGLQNAGRSPTNLAKVKGITQGPNESPSAFLERLKEAYRRYTPYDPEDPGQETNVSMSFIWQSAPDIGRKLERLEDLKSKTLGDLVREAERIFNKGETPEEREERVRRETEEKEERRRAEEEQKEKERDRRRHREMSKLLATVVSGQRQDRQGGERRRPQLDKDQCAYCKEKGHWAKDCPKKPRGPRGPRPQTSLLTLDD